(19) United States
(12) Patent Application Publication
(10) Pub. No.: US 2023/0066152 A1
(43) Pub. Date: Mar. 2, 2023

(54) METHODS TO CHARACTERIZE ENZYMES FOR GENOME ENGINEERING

(71) Applicant: The General Hospital Corporation, Boston, MA (US)

(72) Inventors: Benjamin Kleinstiver, Boston, MA (US); Russell T. Walton, Waban, MA (US)

(21) Appl. No.: 17/794,520

(22) PCT Filed: Jan. 25, 2021
(86) PCT No.: PCT/US2021/014887

§ 371 Date: Jul. 21, 2022

Related U.S. Application Data
(60) Provisional application No. 62/965,645, filed on Jan. 24, 2020.

(51) Int. Cl. C12N 15/10; C12N 15/11; C12N 9/22

(52) U.S. Cl. CPC C12N 15/1068; C12N 15/1086; C12N 15/11; C12N 15/111; C12N 9/22; C12N 2310/20; C12N 2800/80

(57) ABSTRACT

The disclosure provides methods for the concurrent assessment of large numbers of genome engineering proteins, including CRISPR nucleases and base editors. Specifically, the disclosure provides methods of providing a plurality of individual discrete samples comprising populations of cells, wherein each population of cells overexpresses both (i) a single genome engineering protein or a variant thereof and (ii) a reporter protein, lysing the cells to release the proteins; normalizing levels of the genome engineering proteins or variants thereof; allowing the genome engineering proteins or variants thereof to combine with a guide RNA under conditions sufficient to form ribonucleoprotein complexes in each sample; contacting each sample with a plurality of analysis substrates, determining levels of each of the analysis substrate in each sample at a plurality of times; and calculating rate of depletion or enrichment of each of the analysis substrates from each sample.

### CLAIM OF PRIORITY

This application claims priority under 35 USC § 119(e) to U.S. Patent Application Ser. No. 62/965,645, filed on Jan. 24, 2020. The entire contents of the foregoing are hereby incorporated by reference.

### STATEMENT REGARDING FEDERALLY FUNDED RESEARCH

This invention was made with government support under Grant No. CA218870 awarded by the National Institutes of Health. The government has certain rights in the invention.

### FIELD OF THE INVENTION

Described herein are methods for the concurrent assessment of large numbers of genome engineering proteins, including CRISPR nucleases and base editors.

### BACKGROUND

The continued development of genome engineering technologies requires methods that can accurately and rapidly characterize important parameters of these enzymes. Whether through protein engineering to improve the fundamental properties of CRISPR proteins, or through bioinformatic searches to identify previously uncharacterized nucleases, the suite of poorly understood proteins continues to grow. The availability of standardized, accurate, and high-throughput characterization methods is therefore critical to understanding the properties of genome editing technologies.

### SUMMARY

The adaptation of CRISPR-Cas enzymes for genome engineering applications has had a transformational impact on biomedical research. The number of CRISPR-based technologies with different capabilities is rapidly expanding through the discovery of naturally occurring type II (Cas9) and type V (Cas12) orthologs and the engineering of enzymes with improved properties (Makarova et al., Nat. Rev. Microbiol., 18(2):67-83); Anzalone et al., Nat. Biotechnol. 38, 824-844 (2020)). One critical property of these DNA-targeting Cas enzymes is the necessity to recognize a protospacer-adjacent motif (PAM) in their target site (Jinek et al., Science 337, 816-821 (2012)). This requirement fulfills an important biological role, enabling the CRISPR immune system to differentiate self from invading DNA (Marraffini and Sontheimer, Nature 463, 568-571 (2010)). For genome editing applications, the PAM of a Cas protein dictates which genomic sites are accessible to the enzyme. A major bottleneck in the identification or engineering of CRISPR enzymes with unique PAM requirements is the need for scalable experimental methods to characterize PAM preferences in biologically relevant settings. Here, we provide a detailed experimental protocol and steps for analyzing data with HT-PAMDA, a scalable assay to investigate the PAM profiles hundreds of Cas enzymes. Beyond understanding the targeting ranges of Cas enzymes, the HT-PAMDA workflow should be adaptable for scalable characterization of other important properties of CRISPR enzymes including their activities, specificities, guide RNA (gRNA) requirements, and others. For both naturally occurring and optimized enzymes, thorough characterization of the properties of these engineered tools is essential for understanding and benchmarking their performance for genome editing applications.

The present methods include providing a plurality of individual discrete samples comprising populations of cells, preferably mammalian cells, preferably human cells, wherein each population of cells overexpresses both (i) a single genome engineering protein or a variant thereof and (ii) a reporter protein, wherein (i) and (ii) are expressed in a known ratio, preferably 1:1, in the sample; lysing the cells to release the proteins; normalizing levels of the genome engineering proteins or variants thereof based on levels of the reporter protein; combining the genome engineering proteins or variants thereof with a guide RNA (or allowing the proteins or variants to combine with a guide RNA present in the sample) under conditions sufficient to form ribonucleoprotein complexes in each sample; contacting each sample with a plurality of analysis substrates, under conditions sufficient for the genome engineering protein or variant thereof to act on one or more of the substrates; determining levels of each of the analysis substrate in each sample at a plurality of times; and calculating rate of depletion or enrichment of each of the analysis substrates from each sample.

In some embodiments, the genome engineering protein is a nuclease, base editor, or other protein that can alter DNA. In some embodiments, the genome engineering protein can alter the genome of a living cell or genomic DNA in vitro).

In some embodiments, (i) and (ii) are expressed in a known ratio, e.g., 1:1 ratio, from a single nucleic acid construct, preferably a construct comprising a viral 2A sequence in between sequences encoding (i) and (ii), or a direct fusion between sequences encoding (i) and (ii) by a peptide linker.

In some embodiments, the reporter proteins are fluorescent. In some embodiments, expression levels of the reporter proteins is determined by spectrophotometry, image analysis, or other methods to quantify the levels of fluorescence from the reporter protein.

In some embodiments, each different genome engineering protein or variant thereof is expressed in an identified discrete individual population of cells in a single well of a multi-well plate. In some embodiments, a normalized amount of each genome engineering protein is transferred to a second multiwell plate.

In some embodiments, the genome engineering protein is or comprises a CRISPR nuclease, is mixed with a guide RNA to form ribonucleoprotein complexes (or is allowed to form complexes with guide RNAs present in the sample), and is contacted with a population of analysis substrates, each comprising a spacer sequence and a PAM sequence, wherein the population comprises analysis substrates having a plurality of spacer sequences, or plurality of PAM sequences, or both.

In some embodiments, the genome engineering protein is or comprises a cytosine base editor, is mixed with a guide RNA to form ribonucleoprotein complexes, is contacted with a population of analysis substrates, each comprising a spacer sequence and a PAM sequence, wherein the population comprises analysis substrates having a plurality of spacer sequences, or plurality of PAM sequences, or both, and is contacted with an enzyme that converts C-to-U deamination events to double-strand breaks when they co-occur with SpCas9-HNH domain mediated DNA nicks.

In some embodiments, the genome engineering protein is or comprises a adenine base editor, is mixed with a guide RNA to form ribonucleoprotein complexes, is contacted with a population of analysis substrates, each comprising a spacer sequence and a PAM sequence, wherein the population comprises analysis substrates having a plurality of spacer sequences, or plurality of PAM sequences, or both, and is contacted with an enzyme that converts a combination of a target strand nick and a non-target strand deamination event to a double strand break, e.g., Endonuclease V.

In some embodiments, the guide RNA is expressed in the cells along with, or separately from, the Cas protein, or is added to the samples from an exogenous source (e.g., as synthetic or in vitro transcribed RNA).

In some embodiments, the analysis substrates include identifying sequences, preferably 8-10 nt barcodes.

In some embodiments, determining levels of each of the analysis substrate in each sample at a plurality of times comprises using sequencing, detectably labeled probes, arrays, or hybridization methods.

In some embodiments, determining the rate of depletion of each analysis substrate from the population of analysis substrates over time is determined by modeling the depletion as exponential decay and determining the rate constant of depletion for each analysis substrate. In some embodiments, the methods include identifying analysis substrates that are depleted at a faster rate as substrates for the genome engineering protein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE FIGURES

FIG. 1. Schematic of a high-throughput PAM determination assay (HT-PAMDA). a, Schematic of the HT-PAMDA workflow. SpCas9 proteins are expressed in human cells and harvested by gentle lysis, with SpCas9 concentrations normalized by EGFP fluorescence. Two libraries harboring randomized PAMs with separate spacer sequences are subjected to time course in vitro cleavage reactions using SpCas9 lysate complexed with sgRNAs. PAM depletion over time is monitored by deep sequencing and modeled to generate rate constants for each PAM.

FIGS. 2A-B. Reproducibility of the HT-PAMDA. A, Correlation of HT-PAMDA log₁₀ rates (k) for NNNN PAMs across two randomized PAM libraries with distinct spacer sequences (wild-type SpCas9: r²=0.9167; SpCas9-VQR: r²=0.9065). B, Correlation of HT-PAMDA rates for NNNN PAMs across two technical replicates, where each technical replicate is the average of experiments on the two libraries harboring distinct spacer sequences (wild-type SpCas9: r²=0.9770; SpCas9-VQR: r²=0.9329). In panels A and B, HT-PAMDA log₁₀ (k) were set to a minimum value of −4.

FIG. 3. Complete PAM characterizations of SpCas9 variants using HT-PAMDA. HT-PAMDA NNNN profiles of the well-characterized WT SpCas9, SpCas9-VQR, and SpCas9-VRER nucleases. The HT-PAMDA log₁₀ (k) are the mean of at least two replicates against two distinct spacer sequences.

FIGS. 4A-B. Complete PAM characterizations of SpCas9 variants using HT-PAMDA. A, HT-PAMDA characterization of WT, xCas9, SpCas9-NG, and SpG to illustrate their NGNN PAM preferences. The log₁₀ rate constants (k) are the mean of at least two replicates against two distinct spacer sequences. B, HT-PAMDA NNNN profiles of WT SpCas9 and variants: SpG with or without L1111R and A1322R substitutions (top and bottom panels, respectively), SpCas9-NG with or without the requisite L1111R and A1322R substitutions (top and bottom panels, respectively), and xCas9(3.7) with or without the A262T, R324L, S4091, E480K, E543D, and M6941 substitutions (top and bottom panels, respectively). The HT-PAMDA log₁₀ (k) are the mean of at least two replicates against two distinct spacer sequences.

FIG. 5. Characterization of SpCas9 variants bearing systematic substitutions using HT-PAMDA. HT-PAMDA NGNN profiles of WT SpCas9 and engineered variants bearing substitutions at D1135, S1136, G1218, E1219, and T1337; some variants are shown twice for completeness. The HT-PAMDA log₁₀ (k) are the mean of at least two replicates against two distinct spacer sequences.

FIGS. 6A-B. Comparison of HT-PAMDA profiles to human cell activities. A, Modification of 78 endogenous sites in HEK 293T cells bearing NGNN PAMs by WT SpCas9, xCas9, SpCas9-NG, and SpG. Percent modification assessed by targeted sequencing; mean, s.e.m., and individual data points shown for n=3. B, Correlation between HT-PAMDA log₁₀ (k) (see FIGS. 4A and 4B) and mean human cell modification from panel A for each NGNN PAM (WT SpCas9: r²=0.9918; xCas9: r²=0.8715; SpCas9-NG: r²=0.6461; SpG: r²=0.4754). HT-PAMDA log₁₀ (k) were set to a minimum value of −4.

FIG. 7. Workflow of a cytosine base editor high-throughput PAM determination assay (CBE-HT-PAMDA). Schematic of the cytosine base editor (CBE) HT-PAMDA (CBE-HT-PAMDA) workflow. CBE4 max variants are expressed in human cells and harvested by gentle lysis, with CBE4 max concentrations normalized by EGFP fluorescence. A library harboring randomized PAMs is subjected to time course in vitro reactions using CBE4 max lysate complexed with sgRNAs (putative target cytosine bases for deamination within the target site are highlighted in red). Following termination of each reaction, USER enzyme is added to convert C-to-U deamination events to double-strand breaks when they co-occur with SpCas9-HNH domain mediated DNA nicks. PAM depletion overtime is monitored by deep sequencing and modeled to generate rate constants for each PAM.

FIG. 8. NGNN PAM characterizations of CBE variants using CBE-HT-PAMDA. CBE-HT-PAMDA characterization of WT, xCas9, SpCas9-NG, and SpG to illustrate their NGNN PAM preferences. The log₁₀ rate constants (k) are single replicates against one spacer sequences.

FIG. 9. Complete PAM characterizations of CBE variants using CBE-HT-PAMDA. CBE-HT-PAMDA NNNN profiles for WT SpCas9, xCas9, SpCas9-NG, and SpG CBE4 max constructs. CBE-HT-PAMDA log₁₀ (k) values are the from a single replicate against one spacer sequence.

FIG. 10. Comparison of HT-PAMDA and CBE-HT-PAMDA results. For four proteins (WT SpCas9, xCas9, SpCas9-NG, and SpG), we compared the HT-PAMDA values for the nucleases to the CBE-HT-PAMDA values for the CBE variants.

FIG. 11. Workflow of an adenine base editor high-throughput PAM determination assay (ABE-HT-PAMDA). Schematic of the adenine base editor (ABE) HT-PAMDA (ABE-HT-PAMDA) workflow. ABEmax variants are expressed in human cells and harvested by gentle lysis, with ABEmax concentrations normalized by EGFP fluorescence. A library harboring randomized PAMs is subjected to time course in vitro reactions using ABEmax lysate complexed with sgRNAs (the target adenine base for deamination within the target site is highlighted in red). Following termination of each reaction, Endo-V enzyme is added to convert A-to-I deamination events to double-strand breaks when they co-occur with SpCas9-HNH domain mediated DNA nicks. PAM depletion over time is monitored by deep sequencing and modeled to generate rate constants for each PAM.

FIG. 12. Complete PAM characterizations of ABE variants using ABE-HT-PAMDA. ABE-HT-PAMDA NNNN profiles for WT SpCas9, xCas9, SpCas9-NG, and SpG ABEmax constructs. ABE-HT-PAMDA log₁₀ (k) values are the from a single replicate against one spacer sequence.

FIG. 13. Workflow of the spacer mismatch depletion assay. Schematic of the spacer mismatch depletion assay (SPAMDA) used to characterize single mismatch tolerance of intolerance of CRISPR-Cas proteins. SpCas9, Cas12a, or other CRISPR proteins are purified using affinity chromatography; the sgRNA or crRNA can be produced by in vitro transcription or synthesized commercially. A plasmid library harboring all possible single nucleotide substitutions for a given target site (encoded on separate plasmid substrates) is subjected to time course in vitro reactions using the complexed CRISPR-Cas ribonucleoprotein (mismatched bases within the target site are highlighted in red across several panels in the schematic). The depletion of perfectly matched substrates and those harboring single nucleotide mismatches are monitored over time by deep sequencing, followed by modeling as exponential decay to generate rate constants for each substrate.

FIGS. 14A-C. Spacer mismatch tolerance of SpCas9 and engineered variants. A-C, Mismatch tolerance of wild-type (WT) SpCas9, SpCas9-HF1 (bearing N497A/R661A/Q695A/Q926A substitutions), and eSpCas9(1.1) (bearing K848A/K1003A/R1060A substitutions) using the spacer mismatch depletion assay (SPAMDA) across 3 target sites using the same SPAMDA library (targets 1-3 in panels A-C). Reactions were performed at 20° C. and timepoints were taken at 30 seconds, 2 minutes, 8 minutes, and 32 minutes. The sequence of the SPAMDA library is shown on top; target sites are highlighted above the SPAMDA plots with the PAM shown in pink and the spacer of the target site in yellow. The rate of cleavage of a particular substrate is colored, with more rapid cleavage colored in dark blue. Individual squares represent depletion rates for each matched or single-mismatch substrate, colored by rate of depletion (across a gradient of most rapid cleavage in dark blue to slower cleavage in white). The depletion rate of each square corresponding to the base of the matched sequence is the depletion rate of the perfectly matched substrate. n1-n10 represent the 10 negative control substrates bearing multiple substitutions, insertions, or deletions.

FIGS. 15A-B. Spacer mismatch tolerance of AsCas12a and engineered variants. A,B, Mismatch tolerance of wild-type AsCas12a (WT), AsCas12a-HF1 (bearing an N282A substitution), enAsCas12a (bearing E174R/S542R/K548R substitutions), and enAsCas12a-HF1 (bearing N282A/E174R/S542R/K548R substitutions) using the spacer mismatch depletion assay (SPAMDA) across 2 target sites using the same SPAMDA library (targets 1 and 2 in panels A and B, respectively). Reactions were performed at 37° C. and timepoints were taken at 30 seconds, 2 minutes, 8 minutes, and 32 minutes. The sequence of the SPAMDA library is shown on top; target sites are highlighted above the SPAMDA plots with the PAM shown in pink and the spacer of the target site in yellow. The rate of cleavage of a particular substrate is colored, with more rapid cleavage colored in dark blue. Individual squares represent depletion rates for each matched or single-mismatch substrate, colored by rate of depletion (across a gradient of most rapid cleavage in dark blue to slower cleavage in white). The depletion rate of each square corresponding to the base of the matched sequence is the depletion rate of the perfectly matched substrate. n1-n10 represent the 10 negative control substrates bearing multiple substitutions, insertions, or deletions.

FIG. 16. Workflow of the high-throughput spacer mismatch depletion assay. Schematic of the high-throughput spacer mismatch depletion assay (HT-SPAMDA) used to characterize single mismatch tolerance of intolerance of CRISPR-Cas proteins. SpCas9, Cas12a, or other CRISPR proteins are expressed in human cells and harvested by gentle lysis, with concentrations normalized by EGFP fluorescence; the sgRNA or crRNA can be produced by in vitro transcription or synthesized commercially. A plasmid library harboring all possible single nucleotide substitutions for a given target site (encoded on separate plasmid substrates) is subjected to time course in vitro reactions using the complexed CRISPR-Cas ribonucleoprotein (mismatched bases within the target site are highlighted in red across several panels in the schematic). The depletion of perfectly matched substrates and those harboring single nucleotide mismatches are monitored by over time by deep sequencing, followed by modeling as exponential decay to generate rate constants for each substrate.

FIG. 17. High-throughput spacer mismatch tolerance of AsCas12a. Mismatch tolerance of wild-type AsCas12a (WT) using the high-throughput spacer mismatch depletion assay (HT-SPAMDA) across 2 target sites using the same SPAMDA library (targets 1 and 2 in top and bottom panels, respectively). Reactions were performed at 20° C. and timepoints were taken at 30 seconds, 2 minutes, 8 minutes, and 32 minutes. The sequence of the SPAMDA library is shown on top; target sites are highlighted above the SPAMDA plots with the PAM shown in pink and the spacer of the target site in yellow. The rate of cleavage of a particular substrate is colored, with more rapid cleavage colored in dark blue. Individual squares represent depletion rates for each matched or single-mismatch substrate, colored by rate of depletion (across a gradient of most rapid cleavage in dark blue to slower cleavage in white). The depletion rate of each square corresponding to the base of the matched sequence is the depletion rate of the perfectly matched substrate. n1-n10 represent the 10 negative control substrates bearing multiple substitutions, insertions, or deletions.

FIG. 18. Overview of an exemplary HT-PAMDA workflow described in Example 6. The HT-PAMDA protocol enables molecular characterization of the PAMs of different Cas enzymes. The workflow is divided into four major segments: (1) preparation of reagents, including the plasmid libraries harboring randomized PAMs, the gRNA(s), and the human cell lysates that contain Cas enzymes and EGFP (see protocol steps 1-78); (2) performing in vitro cleavage reactions using the reagents generated in section 1, stopping reactions at various timepoints (see protocol steps 79-87); (3) library preparation of the samples generated during the in vitro cleavage reactions of section 2 (the samples are barcoded, amplified, and pooled based on the Cas enzyme, spacer sequence, and timepoint; see protocol steps 88-106); and (4) sequencing of the libraries, data analysis, and visualization (see protocol steps 107-116).

FIG. 19. Detailed exemplary experimental workflow for in vitro cleavage reactions and library preparation as described in Example 6. Stage 1: The gRNA is complexed with the Cas enzymes within the normalized lysates at 37° C., and in vitro timecourse cleavage reactions commence when the substrate library is added. Two substrate libraries (and corresponding gRNAs) harboring distinct spacer sequences are used as technical replicates and to account for sequence-specific effects within the spacers. Aliquots of in vitro cleavage reactions are removed at each timepoint and mixed with pre-aliquoted reaction stop buffer in separate plates to halt the reactions. This process is repeated for all samples (for simplicity, 12 samples per library are shown; the process scales easily to 96 samples per library in a complete plate). Stage 2: Samples are barcoded during PCR #1 with the sample barcoding primers (sBCs) in the first step of library preparation. A given sample receives the same P5 and P7 barcodes across timepoints and substrate libraries. Stage 3: All samples from a timepoint are pooled to create the timepoint pools, which are subsequently barcoded with timepoint barcodes (tBCs) during PCR #2 using standard Illumina P5 and P7 barcoding primers. Stage 4: The timepoint pools are combined to generate the final sequencing-ready HT-PAMDA library.

FIGS. 20A-D. Representations of Cas enzyme PAM preference. a-d, The PAM requirements of wild-type (WT) SpCas9, SpG, and SpRY are represented using four common methods that convey varying degrees of information (sequence preferences, positional dependencies, and absolute activities): plain text (a), sequence logos (generated using Logomaker³⁰; b), PAM wheels (generated using modified Krona plots²⁶; c), and heatmaps (d). All representations of PAM preference were generated using the same HT-PAMDA characterizations, with two replicates on each of two spacer sequences for a total of four replicates per nuclease.

FIGS. 21A-D. Expected results of an HT-PAMDA experiment. a, The representation of each of the 256 4nt PAMs in the substrate library from least to most abundant based on raw read counts. The orange dashed line represents the expected proportion of each PAM if the library were evenly distributed. The narrow distribution of 4 nt PAMs in the untreated substrate library reflects a balanced library; no deviation from the untreated library after 32 minutes is observed in the no-guide control sample. Deviation of the 4 nt PAM distributions with wild-type (WT) SpCas9 after 32 minutes of cleavage reflects depletion of PAMs from the library. A single replicate on a single spacer is plotted for each nuclease. b, Depletion ranges for a selected group of 4 nt PAMs (NGGN, NAGN, NGAN, and NCCN) for WT SpCas9 over time (left panel; mean of the 32 individual PAMs of each category for a single replicate on a single spacer sequence and 95% confidence interval in solid and dotted lines, respectively, of normalized percent PAM remaining for each of the four PAM groups). The counts of PAMs at each timepoint are normalized and HT-PAMDA rate constants are calculated and used to generate the heatmap visualization (right panel). The heatmap visualization represents the mean depletion rates of two replicates on each of two spacer sequences for a total of four replicates. c, Scatterplot comparing technical replicate HT-PAMDA experiments with WT SpCas9, SpG, and SpRY. Each point represents a 4 nt PAM. Each replicate value is the average of two separate experiments using two substrate libraries harboring distinct spacer sequences. d, Scatterplot comparing replicate HT-PAMDA experiments with WT SpCas9, SpG, and SpRY on substrate libraries harboring two distinct spacer sequences. Each point represents a single replicate on each spacer library for a 4 nt PAM. HT-PAMDA log₁₀ rates were set to a minimum value of −5 (panels c and d).

### DETAILED DESCRIPTION

Here we describe a series of methods that enable the concurrent assessment of large numbers of genome engineering proteins, e.g., CRISPR nucleases and base editors, at a scale not previously performed, to reduce or eliminate the bottleneck of enzyme characterization in projects that seek to discover or engineer new Cas variants. The assay differentiates itself from prior methods at least because it can be executed in high-throughput format in a human cell lysate, with facile quantification and normalization of the expressed protein of interest (a step critical for accurate property assessment). These methods can be adapted to study different properties (e.g. the PAM preferences, mismatch tolerance, or general specificities) of many CRISPR proteins including nucleases, cytosine base editors (CBEs)¹, or adenine base editors (ABEs)².

### High-Throughput Assays

The methods described herein include the use of cultured mammalian cells, preferably human cells, that have been engineered to overexpress both (i) a genome engineering protein (e.g., nuclease, base editor, or other protein that can alter DNA, e.g., can alter the genome of a living cell or genomic DNA in vitro) or a variant thereof and (ii) a reporter protein. In preferred embodiments, (i) and (ii) are expressed in a known, fixed ratio, preferably a 1:1 ratio, e.g., from a single nucleic acid construct, e.g., as a fusion protein (e.g., with an intervening linker sequence) a construct comprising a viral 2A sequence in between sequences encoding (i) and (ii). See, e.g., Lewis et al., J. Neuroscience Methods, 256:22-29 (2015). In some embodiments, the cells are also engineered to express a guide RNA.

In preferred embodiments, each different genome engineering protein or variant thereof is expressed in an identified discrete individual population of cells, optionally in a single well of a multi-well plate. The cells are then lysed and expression levels of the proteins determined, e.g., by spectrophotometry, image analysis, or other methods to quantify the levels of fluorescence or signal from the reporter protein. A normalized amount of each protein is then transferred to a second container, e.g., a second multiwell plate, mixed with a guide RNA or prime template to form ribonucleoprotein complexes, and contacted with a population of analysis substrates; in some embodiments, the gRNA can be co-expressed in the cells rather than added later. For example, gRNA expression plasmids can be co-transfected in a molar excess with of the nuclease expression plasmid such that the cell lysate will contain complexed RNPs. This step can be performed to avoid large numbers of in vitro transcription reactions to produce gRNAs. Then amounts of the analysis substrate in the sample are determined at one, two, three, or more time points and the rate of depletion of each analysis substrate from the population of analysis substrates overtime is determined, e.g., via modeling the depletion as exponential decay; the rate constant of depletion for each analysis substrate (e.g., for each PAM sequence) is then used to calculate comprehensive preferences (e.g., PAM preferences) for each variant.

Genome Engineering Proteins

In some embodiments, the methods include expressing a CRISPR nuclease or CRISPR-nuclease based genome editing reagent, e.g., Cas9 or a related protein, a base editor, or a prime editor, or a variant thereof. A number of such reagents, and methods for creating variants, are known in the art. In some embodiments, the protein is or comprises SaCas9, SpCas9, or another CRISPR-Cas protein, including other Cas9 orthologs (Esvelt et al., Nature Methods, 10(11):1116-21; Fonfara et al., Nucleic Acids Res., 42:2577-2590) with various levels of basal activity (e.g. SaCas9 (Ran et al., Nature, 520(7546):186-91; Kleinstiver et al., Nature, 523(7561)481-5; Kleinstiver et al., Nature Biotechnology, 33(12):1293-1298), St1Cas9 (Deveau et al., J. Bacteriol., 190:1390-1400; Horvath, et al., J. Bacteriol., 190:1401-1412; Kleinstiver et al., Nature, 523(7561)481-5), St3Cas9 (Gasiunas et al., Proc. Natl. Acad. Sci. USA, 109(39):E2579-86), NmeCas9 (Hou et al., Proc. Natl. Acad. Sci. USA, 110(39):15644-9), Nme2Cas9 (Edraki et al., Molecular Cell, 73(4):714-726.e4), CjeCas9 (Kim et al., Nature Communications, (8)14500), and other Cas9 orthologs; Cas12a orthologs (Zetsche et al., Cell, 163(3):759-71; Zetsche et al., doi.org/10.2302/kjm.2019-0009-OA), and other Cas3 (HidalgoCantabrana, PMID: 31922192, DOI: 10.1042/BST20190119), Cas12 (Koonin et al. Curr. Opp. Micro., 37:67-68); Yan et al., Science, 363(6422):88-91), Cas13 (Abudayyeh et al., Science, 353(6299):aaf5573; Shmakov et al., Molecular Cell, 60(3):385-97; Abudayyeh et al., Nature, 550(7675):280-284), Cas14 proteins (Harrington et al., Science, 362(6416):839-842), and those collectively reviewed in Makarova et al. (Nat. Rev. Microbiol., 18(2):67-83), as well as engineered variants thereof, which can be used alone or incorporated into a non-nuclease construct, e.g., a nickase (Mali et al., Nature Biotechnology, 31(9):833-8; Ran et al. Cell, 154(6):1380-9), Fokl-dCas9 fusions (Tsai et al., Nature Biotechnology, 32(6):569-76); Guilinger et al., Nature Biotechnology, 32(6):577-582), a base editor (Komor et al. Nature, 533(7603):420-4; Gaudelli et al. Nature, 551(7681):464-471; Rees et al., Nat. Rev. Genet., 19(12):770-788), or a prime editor (Anzalone et al., Nature, 576(7785):149-157).

In some embodiments, the variant is at least 50, 60, 65, 70, 75, 80, 85, 90, 95, or 99% identical to a wild type or reference sequence, and/or comprises at least 1, 2, 3, 4, 5, 6, 7, 8, 9, 10, 11, 12, 13, 14, 15, 16, 17, 18, 19, or 20 mutations/substitutions, e.g., up to 1%, 2%, 2%, 3%, 4%, 5%, 10%, 15%, 20%, 25%, 30%, 35%, 40%, 45%, or 50% of the sequence, as compared to the wild type or reference sequence. The variants can be random mutations, or can be introduced using a rational design approach to alter one or more characteristics of the protein (e.g., on target effects, off target effects, PAM specificity, and so on). In some embodiments, the mutation is a conservative substitution, e.g., including substitutions within the following groups: glycine, alanine; valine, isoleucine, leucine; aspartic acid, glutamic acid, asparagine, glutamine; serine, threonine; lysine, arginine; and phenylalanine, tyrosine. In some embodiments, the mutation is a non-conservative substitution. One of skill in the art could identify and generate such variants.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher PIus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned using the BLAST algorithm and the default parameters.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Reporter Proteins

A number of reporter proteins are known in the art, and include green fluorescent protein (GFP), variant of green fluorescent protein (GFP10), enhanced GFP (eGFP), TurboGFP, GFPS65T, TagGFP2, mUKGEmerald GFP, Superfolder GFP, GFPuv, destabilised EGFP (dEGFP), Azami Green, mWasabi, Clover, mClover3, mNeonGreen, NowGFP, Sapphire, TSapphire, mAmetrine, photoactivatable GFP (PA-GFP), Kaede, Kikume, mKikGR, tdEos, Dendra2, mEosFP2, Dronpa, blue fluorescent protein (BFP), eBFP2, azurite BFP, mTagBFP, mKalamal, mTagBFP2, shBFP, cyan fluorescent protein (CFP), eCFP, Cerulian CFP, SCFP3A, destabilised ECFP (dECFP), CyPet, mTurquoise, mTurquoise2, mTFPI, photoswitchable CFP2 (PS-CFP2), TagCFP, mTFP1, mMidoriishi-Cyan, aquamarine, mKeima, mBeRFP, LSS-mKate2, LSS-mKatel, LSS-mOrange, CyOFP1, Sandercyanin, red fluorescent protein (RFP), eRFP, mRaspberry, mRuby, mApple, mCardinal, mStable, mMaroonl, mGarnet2, tdTomato, mTangerine, mStrawberry, TagRFP, TagRFP657, TagRFP675, mKate2, HcRed, t-HcRed, HcRed-Tandem, mPlum, mNeptune, NirFP, Kindling, far red fluorescent protein, yellow fluorescent protein (YFP), eYFP, destabilised EYFP (dEYFP), TagYFP, Topaz, Venus, SYFP2, mCherry, PA-mCherry, Citrine, mCitrine, Ypet, IANRFP-AS83, mPapayal, mCyRFP1, mHoneydew, mBanana, mOrange, Kusabira Orange, Kusabira Orange 2, mKusabira Orange, mOrange 2, mKO.sub.K, mKO2, mGrapel, mGrape2, zsYellow, eqFP611, Sirius, Sandercyanin, shBFP-N158S/L1731, near infrared proteins, iFP1.4, iRFP713, iRFP670, iRFP682, iRFP702, iRFP720, iFP2.0, mlFP, TDsmURFP, miRFP670, Brilliant Violet (BV) 421, BV 605, BV 510, BV 711, BV786, PerCP, PerCP/Cy5.5, DsRed, DsRed2, mRFPI, pocilloporin, Renilla GFP, Monster GFP, paGFP, or a Phycobiliprotein, or a biologically active variant or fragment of any one thereof.

Cells

The methods described herein include expression in cells, e.g., mammalian cells, preferably human cells, e.g., cultured cells. Exemplary human cultured cell lines include 3T3; A375; A431; A549; Daudi; HEK293; HeLa; HepaRG; HepG2; Jurkat; MDA-MB-231; MDA-MB436; MDA-MB-468; Saos-2; 1321N1; AtT-20; B16; Ba/F3; BHK; Caki; Calu; CHO; COS; CV-1; Detroit; DMS; EPH4; HEK293T; HL-60; HUVEC; K562; Kasumi; LLC-MK2; MCF; MDA-MB; MDCK; PC3 (PC-3); Phoenix; SCC; Sf21; Sf9; SNU; T47D; THP1; U937 (U-937); U2-OS; and Vero cells.

Methods for expressing proteins in cells are well known in the art. Typically, the cells are combined with an exogenous nucleic acid sequence encoding the proteins and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection, and electroporation.

### High-Throughput PAM Determination Assay (HT-PAMDA) for Nucleases

For PAM specificity analysis, variants designed to have or suspected to have different PAM preferences are expressed in cells and normalized as described above. The analysis substrates comprise a library of oligonucleotides, each comprising a spacer sequence that corresponds to the spacer sequence of the guide RNA and one of a plurality of different PAM sequences. The rate of depletion of each analysis substrate from the population of analysis substrates due to the action of the nuclease overtime is determined, e.g., via modeling the depletion as exponential decay; the rate constant of depletion for each analysis substrate (and thus for each PAM sequence) is then used to calculate comprehensive PAM preferences for each variant.

While our initial implementation of HT-PAMDA was to profile the PAM preferences of SpCas9 variants, this approach should be extensible to other Cas enzymes and for the in vitro characterization of other properties. The enzyme-containing lysate and/or the PAM library (substrate library) can be substituted to develop new protocols to understand other parameters beyond targeting range. As examples, two alternate implementations to characterize the PAM requirements of C-to-T base editors (CBEs) and A-to-G base editors (ABEs) are highlighted in the CBE-HT-PAMDA and ABE-HT-PAMDA protocols, respectively. In these assays, the lysates containing normalized Cas nucleases are substituted for CBEs or ABEs to characterize the PAM requirements of these enzymes that nick and deaminate DNA compared to nucleases that generate double-strand breaks (Komor et al., Nature 533, 420-424 (2016); Gaudelli et al., Nature 551, 464-471 (2017)). Pending appropriate modifications (discussed below), the HT-PAMDA method is applicable to study other Cas9 orthologs and Cas proteins of different classes (such as Cas12a proteins, as we demonstrated with the lower-throughput PAMDA approach)(Kleinstiver et al., Nat. Biotechnol. 37, 276-282 (2019)). Alternatively, the protocol can also be modified to study different properties of Cas proteins. For example, the target specificities of Cas proteins can be studied using this method by substituting the randomized PAM substrate libraries for libraries encoding spacer sequences with mismatched bases. Broadly, HT-PAMDA and similar adaptations can form a suite of methods for the rapid characterization of the properties of genome editing tools.

### Cytosine Base Editor High-Throughput PAM Determination Assay (CBE-HT-PAMDA)

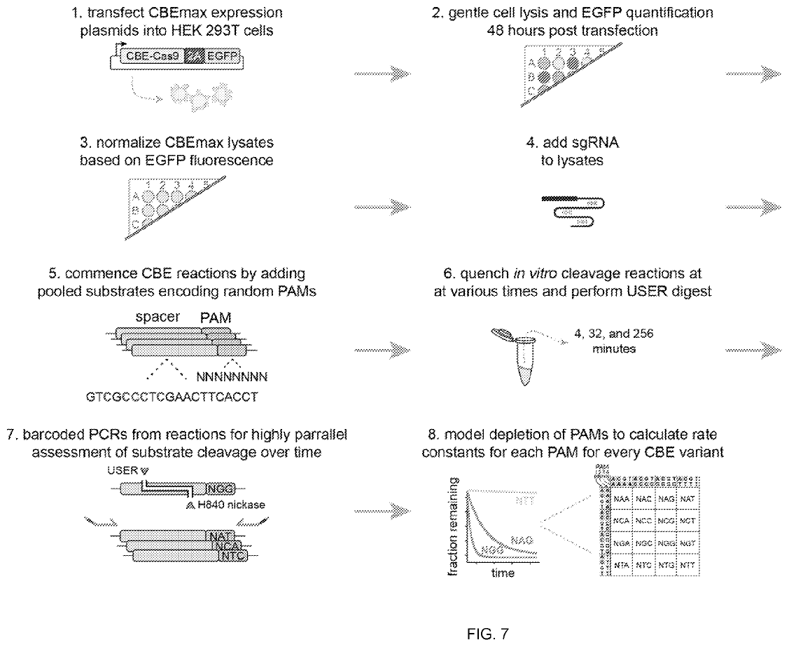

To assess whether SpCas9 nucleases and BEs exhibit consistent PAM profiles, the HT-PAMDA assay described above was adapted to function in the absence of SpCas9-mediated DNA cleavage. Instead of double-strand DNA cleavage by SpCas9, this assay relies on SpCas9-based nicking and deamination of a cytosine by the tethered rAPOBEC1 domain. The combination of a target strand nick and a non-target strand deamination event is later converted to a double strand break using USER enzyme to remove the uracil base and cleave the non-target strand backbone, depleting CBE-targetable PAM-containing substrates from the library. Again, the rate of depletion of each analysis substrate from the population of analysis substrates due to the action of the nuclease overtime is determined, e.g., via modeling the depletion as exponential decay; the rate constant of depletion for each analysis substrate (and thus for each PAM sequence) is then used to calculate comprehensive PAM preferences for each variant. See, e.g., FIG. 7 and Example 2.

### Adenine Base Editor High-Throughput PAM Determination Assay (ABE-HT-PAMDA)

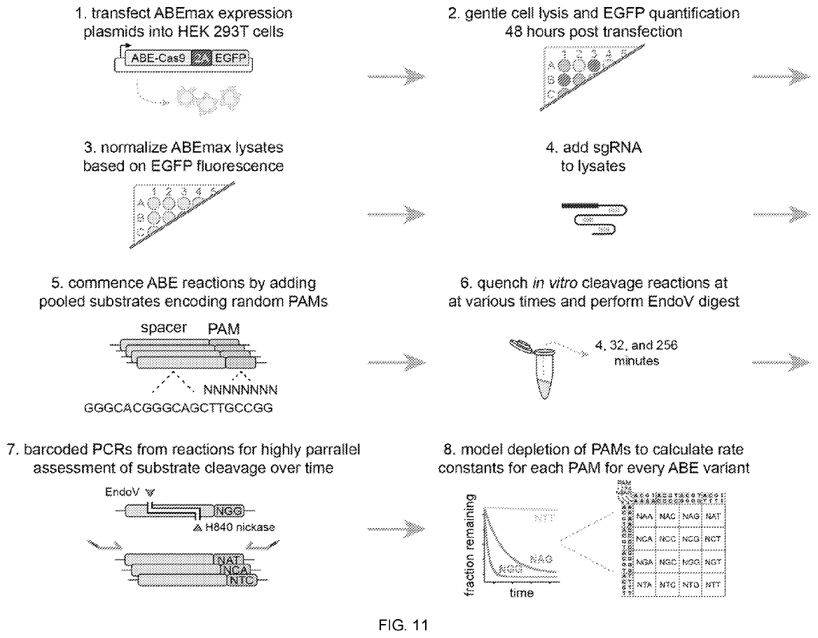

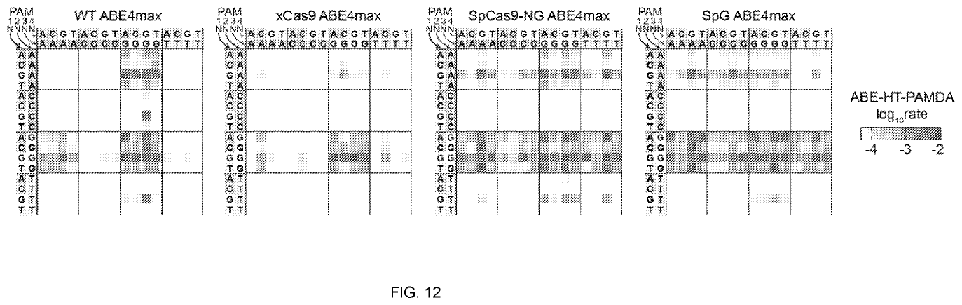

Adenine base editors (ABEs) enable the generation of A-to-G mutations in human cells². To characterize the PAM preferences of ABEs, an adenine base editor high-throughput PAM determination assay (ABE-HT-PAMDA) was developed. Rather than relying on cleavage of both DNA strands by SpCas9 to deplete sequences as in HT-PAMDA (Example 1), ABE-HT-PAMDA relies on SpCas9 nicking of the target strand and deamination of an adenine to inosine in the non-target strand by the TadA domains of the ABE². During the in vitro ABE-HT-PAMDA protocol, the combination of a target strand nick and a non-target strand deamination event is later converted to a double strand break using Endonuclease V (NEB) to nick the non-target strand at the second phosphodiester bond 3′ of the inosine. Again, the rate of depletion of each analysis substrate from the population of analysis substrates due to the action of the nuclease over time is determined, e.g., via modeling the depletion as exponential decay; the rate constant of depletion for each analysis substrate (and thus for each PAM sequence) is then used to calculate comprehensive PAM preferences for each variant. See, e.g., FIG. 11 and Example 3.

### Single Nucleotide Specificity Characterization Assay for Nucleases-Spacer Mismatch Depletion Assay (SPAMDA) and High-Throughput SPAMDA (HT-SPAMDA)

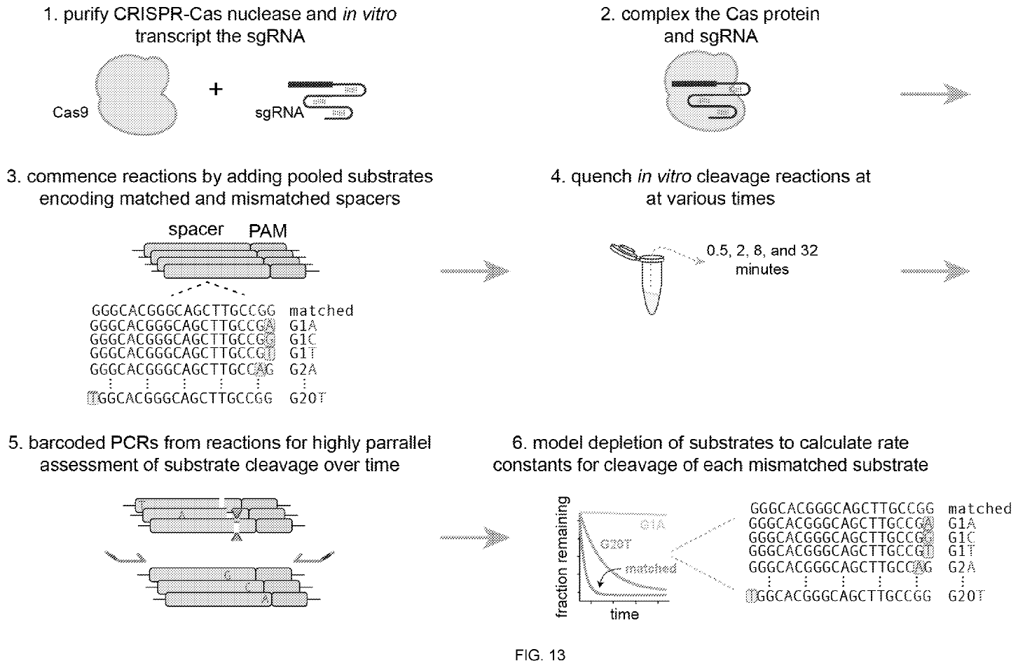

Assays that enable the rapid profiling of the tolerance of Cas9 and Cas12a enzymes to single nucleotide substitutions in their target site were developed. The assays are technically similar to the PAMDA (Example 1) but instead of establishing PAM preferences enable thorough characterization of single mismatch tolerance. Thus, in place of using a library of substrates encoding random PAM sequences, we designed and constructed a spacer mismatch depletion assay (SPAMDA) library containing a perfectly matched substrate, those bearing all possible single substitutions across a 39 nt sequence, and 10 controls bearing multiple substitutions, insertions, or deletions (see FIG. 13 panel 3 and Methods). Each substrate of the library also encodes a unique 8 nt barcode to enable identification of each substrate irrespective of sequencing errors (that might generate erroneous single nt mismatch calls). This library of plasmids is then used as a substrate for in vitro cleavage reactions with purified Cas9, Cas12a, or other CRISPR proteins. Additionally, the library is designed with multiple PAM sequences of common CRISPR enzymes (NGG (3′) for SpCas9, NNGRRT (3′) for SaCas9, and TTTV (5′) for Cas12a orthologs) falling within in the 39 nt sequence to enable characterization of multiple nucleases, each with multiple spacer sequences, all with a single library (FIG. 13). In SPAMDA, a constant amount of the normalized purified protein is utilized in time-course in vitro cleavage reactions of the libraries. Targeted sequencing of the cleavage reactions at various time points allows quantitation of the rate of depletion of each spacer substrate from the population over time; the rate constant for each matched or mismatched substrate therefore enables us to determine a comprehensive single nt specificity profile for each Cas9 or Cas12a variant. See Example 4, FIG. 13.

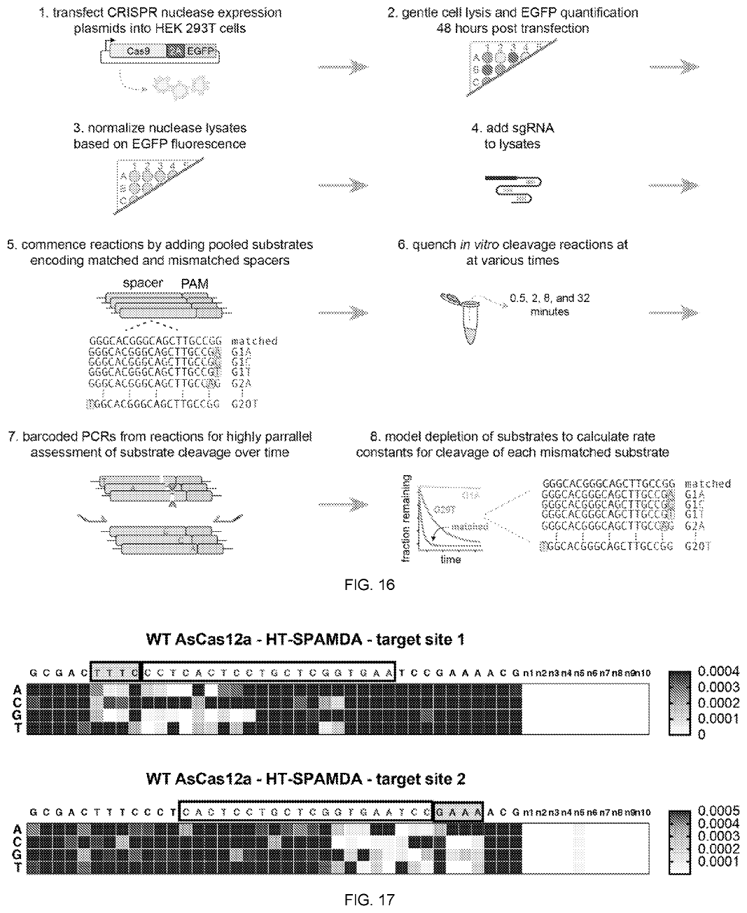

The high throughout version of this assay utilizes the same SPAMDA library bearing all single mismatches across a 39 nt sequence, but instead of purified protein the HT assay utilizes human cell lysates containing expressed CRISPR proteins (as done for the HT-PAMDA assays, see Example 1). The variable expression of Cas9 or Cas12a proteins across different transfections is linked to the expression of a 2A-EGFP fluorescence, permitting the normalization of nuclease concentrations based on a fluorescein standard curve (FIG. 16). Then, a constant amount of the normalized human cell lysates are then subject to time-course in vitro cleavage reactions of the SPAMDA libraries, with quantification of matched or mismatched substrate depletion enabling us to determine a comprehensive single nt specificity profile for each variant. See Example 5, FIG. 16.

In each method, the rate of depletion of each analysis substrate from the population of analysis substrates due to the action of the nuclease over time is determined, e.g., via modeling the depletion as exponential decay; the rate constant of depletion for each analysis substrate (and thus for each spacer sequence) is then used to calculate comprehensive single mismatch tolerances for each variant.

### Non-CRISPR Genome Editing Proteins

Although the above have been described with regard to CRISPR nucleases, CRISPR-nuclease based constructs, and CRISPR base editors, the methods can also be applied to high throughput analysis of sequence specificity of other classes of genome editing proteins (including other CRISPR derivatives, including nickases, prime editors, and others). For example, this strategy can be applied to other nucleic acid-binding proteins (zinc-fingers and zinc-finger nucleases (ZFs and ZFNs), transcription activator-like effectors and transcription activator-like effector nucleases (TALEs and TALENs), restriction enzymes, transposases, recombinases, integrases, etc., using analysis substrate libraries suitable for the protein to be analyzed.

### EXAMPLES

### Methods

The following materials and methods were used in the Examples below.

### Methods for HT-PAMDA Assays

High-throughput PAM Determination assay for nucleases

The high-throughput PAM determination assay (HT-PAMDA) was performed using linearized randomized PAM-containing plasmid substrates that were subject to in vitro cleavage reactions with SpCas9 and variant proteins. First, SpCas9 ribonucleoproteins (RNPs) were complexed by mixing 4.375 μL of normalized whole-cell lysate (150 nM Fluorescein) with 8.75 pmol of in vitro transcribed sgRNA and incubating for 5 minutes at 37° C. Cleavage reactions were initiated by the addition of 43.75 fmol of randomized-PAM plasmid library and buffer to bring the total reaction volume to 17.5 μL with a final composition of 10 mM Hepes pH 7.5, 150 mM NaCl, and 5 mM MgCl₂. Reactions were performed at 37° C. and aliquots were terminated at timepoints of 1, 8, and 32 minutes by removing 5 μL aliquots from the reaction and mixing with 5 μL of stop buffer (50 mM EDTA and 2 mg/ml Proteinase K (NEB)), incubating at room temperature for 10-minutes, and heat inactivating at 98° C. for 5 minutes. For all variants characterized, time courses were completed on both libraries harboring distinct spacer sequences for n=2; several variants were characterized with additional replicates to evaluate reproducibility of the assay, where for those variants the final data is an average of all replicates.

Next, approximately 3 ng of digested PAM library for each SpCas9 variant and reaction timepoint was PCR amplified using Q5 polymerase (NEB) and barcoded using unique combinations of the i5 and i7 primers. PCR products were pooled for each time point, purified using paramagnetic beads, and prepared for sequencing using one of two library preparation methods. Pooled amplicons were prepared for sequencing using either (1) the KAPA HTP PCR-free Library Preparation Kit (KAPA BioSystems), or (2) a PCR-based method where pooled amplicons were treated with Exonuclease I, purified using paramagnetic beads, amplified using Q5 polymerase and primers with approximately 250 pg of pooled amplicons at template, and again purified using paramagnetic beads. Libraries constructed via either method were quantified using the Universal KAPA Illumina Library qPCR Quantification Kit (KAPA Biosystems) and sequenced on a NextSeq sequencer using a either 150-cycle (method 1) or 75-cycle (method 2) NextSeq 500/550 High Output v2.5 kits (Illumina). Identical cleavage reactions prepared and sequenced via either library preparation method did not exhibit substantial differences.

Sequencing reads were analyzed using a custom Python script to determine cleavage rates for all SpCas9 nucleases on each substrate with unique spacers and PAMs, similar to as previously described³⁶. Briefly, reads were assigned to specific SpCas9 variants based on based on custom pooling barcodes, assigned timepoints based on the combination of i5 and i7 primer barcodes, assigned to a plasmid library based on the spacer sequence, and assigned to a 3 (NNNN) or 4 (NNNN) nt PAM based on the identities of the DNA bases adjacent to the spacer sequence. Counts for all PAMs were computed for every SpCas9 variant, plasmid library, and timepoint, corrected for inter-sample differences in sequencing depth, converted to a fraction of the initial representation of that PAM in the original plasmid library (as determined by an untreated control), and then normalized to account for the increased fractional representation of uncut substrates over time due to depletion of cleaved substrates (by selecting the five PAMs with the highest average fractional representation across all time points to represent the profile of uncleavable substrates). The depletion of each PAM over time was then fit to an exponential decay model (y(t)=Ae^{-kt}, where y(t) is the normalized PAM count, t is the time (seconds), k is the rate constant, and A is a constant), by nonlinear regression. Reported rates are the average across both spacer sequences and across technical replicates when performed. Nonlinear least squares curve fitting was utilized to model Cas9 nuclease and CBE activities, whereas linear least squares curve fitting was previously used for our Cas12a PAMDA assay⁶.

### CBE-HT-PAMDA

The cytosine base editor high-throughput PAM determination assay (CBE-HT-PAMDA) was performed using a linearized randomized PAM-containing plasmid library that was subjected to in vitro reactions with base editor variants. First, base editor proteins were complexed with sgRNAs by mixing 8.75 μL of normalized whole-cell lysate (300 nM Fluorescein) with 14 pmol of in vitro transcribed sgRNA and incubating for 5 minutes at 37° C. Cleavage reactions were initiated by the addition of 43.75 fmol of randomized-PAM plasmid library and buffer to bring the total reaction volume to 17.5 μL with a final composition of 10 mM Hepes pH 7.5, 150 mM NaCl, and 5 mM MgCl₂. Reactions were performed at 37° C. and aliquots were terminated at timepoints of 4, 32, and 256 minutes by removing 5 μL aliquots from the reaction and mixing with 5 μL of stop buffer (50 mM EDTA and 2 mg/ml Proteinase K (NEB)), incubating at room temperature for 10-minutes, and heat inactivating at 98° C. for 5 minutes. Deamination and nicking events were converted to double strand breaks through the addition of 1 unit of USER enzyme (NEB) in 5 μL of 1× NEB buffer 4 to each reaction, bringing the total volume to 15 μL. After an hour incubation at 37° C., reactions were stopped by adding of 5 ul of 4 mg/mL Proteinase K in 1 mM Tris pH 8.0, incubating at room temperature for 10-minutes, and heat inactivating at 98° C. for 5 minutes. Reactions were carried out on a single plasmid library for each base editor. Samples were subsequently processed as described above for HT-PAMDA for nucleases, with the exception that depletion rates are for a single spacer sequence for CBE-HT-PAMDA, rather than the average of two spacer sequences as in the nuclease analysis.

### ABE-HT-PAMDA

The high-throughput PAM determination assay for ABEs (ABE-HT-PAMDA) was performed using linearized randomized PAM-containing plasmid substrates that were subject to in vitro reactions with base editor variants. First, base editor proteins were complexed with sgRNAs by mixing 8.75 μl of normalized whole-cell lysate (300 mM Fluorescein) with 14 pmol of in vitro transcribed sgRNA and incubating for 5 minutes at 37° C. Cleavage reactions were initiated by the addition of 43.75 fmol of randomized-PAM plasmid library and buffer to bring the total reaction volume to 17.5 μl with a final composition of 10 mM Hepes pH 7.5, 150 mM NaCl, and 5 mM MgCl2. Reactions were performed at 37° C. and aliquots were terminated at timepoints of 4, 32, and 256 minutes by removing 5 μl aliquots from the reaction and mixing with 5 μl of stop buffer (50 mM EDTA and 2 mg/ml Proteinase K (NEB)), incubating at room temperature for 10-minutes, and heat inactivating at 98° C. for 5 minutes. Deamination and nicking events were converted to double strand breaks through the addition of 5 units of Endonuclease V (NEB) in 5 μl of 1× NEB buffer 4 to each reaction, bringing the total volume to 15 μL. After an hour incubation at 37° C., reactions were stopped by adding of 5 ul of 4 mg/mL Proteinase K in 1 mM Tris pH 8.0, incubating at room temperature for 10-minutes, and heat inactivating at 98° C. for 5 minutes. Reactions were carried out on a single plasmid library for each base editor. Samples were subsequently processed as described above for HT-PAMDA for nucleases.

### Methods for Spacer Mismatch Depletion Assay (Spamda) Protocol

### Plasmids and Oligonucleotides for SPAMDA or HT-SPAMDA

The SPAMDA plasmid library was prepared by pooling individually cloned substrate plasmids. Oligos pairs harboring the 39 base pair target sequence, a unique 8 base pair barcode, and restriction enzyme overhangs were annealed and ligated into the NheI and HindIII sites of BPK1520 (Addgene plasmid 65777). The final SPAMDA library was a 128-plasmid pool consisting of the “on-target” sequence (1 plasmid), all single nucleotide mismatches throughout the 39 base pair sequence (117 plasmids), and 10 negative control plasmids (6 plasmids with 6 substitutions relative to the “on-target”, 2 plasmids with multiple nucleotide insertions, and 2 plasmids multiple nucleotide deletions). Plasmids were pooled in equimolar ratios.

In Vitro Transcription of sgRNAs or crRNAs for SPAMDA

SpCas9 sgRNAs were in vitro transcribed at 37° C. for 16 hours from roughly 1 μg of HindIII linearized sgRNA T7-transcription plasmid template (cloned into MSP3485) using the T7 RiboMAX Express Large Scale RNA Production Kit (Promega). The DNA template was degraded by the addition of 1 μL RQ1 DNase at 37° C. for 15 minutes. sgRNAs were purified with the MEGAclear Transcription Clean-Up Kit (ThermoFisher) and refolded by heating to 90° C. for 5 minutes and then cooling to room temperature for over 15 minutes.

Cas12a crRNAs were in vitro transcribed from roughly 1 μg of HindIII linearized crRNA transcription plasmid (cloned into MSP3491, Addgene plasmid 114067) using the T7 RiboMAX Express Large Scale RNA Production kit (Promega) at 37° C. for 16 h. The DNA template was degraded by the addition of 1 μL RQ1 DNase and digestion at 37° C. for 15 min. Transcribed crRNAs were subsequently purified with the miRNeasy Mini Kit (Qiagen) and refolded by heating to 90° C. for 5 minutes and then cooling to room temperature for over 15 minutes.

### Spacer Mismatch Depletion Assay (SPAMDA)

To perform the spacer mismatch depletion assay, first ribonucleoproteins (RNPs) were formed by complexing 1.8 pmol of purified SpCas9 protein with 3.6 pmol of in vitro transcribed sgRNA or 7.2 pmol of purified AsCas12a protein with 14.4 pmol of in vitro transcribed crRNA and incubating for 5 minutes at 37° C. Reactions were initiated through the addition of 225 fmol of PvuI-linearized SPAMDA plasmid library and buffer to a final composition of 10 mM Hepes pH 7.5, 150 mM NaCl, and 5 mM MgCl₂ in 45 μL. Reactions were incubated at either 37° C. or 20° C. At each timepoint (30 seconds, 2 minutes, 8 minutes, and 32 minutes), 10 μL of reaction mix was transferred into 10 ul of reaction stop buffer (50 mM EDTA and 2 mg/ml Proteinase K (NEB)) and incubated at room temperature for 10 minutes. Terminated reactions were then purified using paramagnetic beads prepared as previously described⁶.

Next, approximately 3 ng of digested SPAMDA library for each reaction timepoint was PCR amplified using Q5 polymerase (NEB) and barcoded using unique combinations of barcoded PCR primers. PCR products were pooled for each time point, purified using paramagnetic beads, and prepared for sequencing using the KAPA HTP PCR-free Library Preparation Kit (KAPA BioSystems). Libraries were quantified using the Universal KAPA Illumina Library qPCR Quantification Kit (KAPA Biosystems) and sequenced on a MiSeq sequencer using a 300-cycle MiSeq Reagent Kit v2 (Illumina).

### High-Throughput Spacer Mismatch Depletion Assay (HT-SPAMDA)

The high-throughput spacer mismatch depletion assay HT-SPAMDA was performed similarly to SPAMDA, but substitutes purified SpCas9 or AsCas12a with unpurified protein in human cell lysate. To generate SpCas9 and AsCas12a proteins from human cell lysates, approximately 20-24 hours prior to transfection 1.5×10⁵ HEK 293T cells were seeded in 24-well plates. Transfections containing 500 ng of human codon optimized nuclease expression plasmid (with a -P2A-EGFP signal) and 1.5 μL TransIT-X2 were mixed in a total volume of 50 μL of Opti-MEM, incubated at room temperature for 15 minutes, and added to the cells. The lysate was harvested after 48 hours by discarding the media and resuspending the cells in 100 ul of gentle lysis buffer (1× SIGMAFAST Protease Inhibitor Cocktail, EDTA-Free (Millipore Sigma), 20 mM Hepes pH 7.5, 100 mM KCl, 5 mM MgCl₂, 5% glycerol, 1 mM DTT, and 0.1% Triton X-100). The amount of nuclease protein was approximated from the whole-cell lysate based on EGFP fluorescence. Lysates were normalized to 150 nM Fluorescein (Sigma) based on a Fluorescein standard curve. Fluorescence was measured in 384-well plates on a DTX 880 Multimode Plate Reader (Beckman Coulter) with λₑₓ=485 nm and λₑₘ=535 nm.

RNPs were then formed by mixing 22.5 pmol sgRNA or crRNA with 11.25 μL of normalized lysate with either SpCas9 or AsCas12a, respectively. Reactions were initiated through the addition of 225 fmol of PvuI-linearized SPAMDA plasmid library and buffer to a final composition of 10 mM Hepes pH 7.5, 150 mM NaCl, and 5 mM MgCl₂ in 45 μL. Reactions were incubated at 37° C. At each timepoint (30 seconds, 2 minutes, 8 minutes, and 32 minutes), 10 μL of reaction mix was transferred into 10 ul of reaction stop buffer (50 mM EDTA and 2 mg/ml Proteinase K (NEB)) and incubated at room temperature for 10 minutes. Terminated reactions were then purified using paramagnetic beads prepared as previously described^{6,21}.

Next, approximately 3 ng of digested HT-SPAMDA library for each reaction timepoint was PCR amplified using Q5 polymerase (NEB) and barcoded using unique combinations of barcoded PCR primers. PCR products were pooled for each time point, purified using paramagnetic beads, and prepared for sequencing using the KAPA HTP PCR-free Library Preparation Kit (KAPA BioSystems). Libraries were quantified using the Universal KAPA Illumina Library qPCR Quantification Kit (KAPA Biosystems) and sequenced on a MiSeq sequencer using a 300-cycle MiSeq Reagent Kit v2 (Illumina).

### Analysis of SPAMDA and HT-SPAMDA

Sequencing reads were analyzed using a custom Python script to determine cleavage rates for each nuclease on each substrate. Briefly, reads were assigned to specific nucleases based on custom pooling barcodes, assigned timepoints based on the combination of i5 and i7 primer barcodes, and assigned to substrate based on the 8 base pair barcode and the 39 base pair target sequence. Counts for all substrates were computed for every nuclease and timepoint, corrected for inter-sample differences in sequencing depth, converted to a fraction of the initial representation of that substrate in the original plasmid library (as determined by an untreated control), and then normalized to account for the increased fractional representation of uncut substrates over time due to depletion of cleaved substrates (by selecting the 10 negative control substrates to represent the profile of uncleavable substrates). The depletion of each substrate over time was then fit to an exponential decay model (y(t)=Ae-kt, where y(t) is the normalized substrate count, t is the time (seconds), k is the rate constant, and A is a constant), by linear regression.

### Example 1. Development of a High-Throughput PAM Characterization Assay for CRISPR Nucleases

The protospacer-adjacent motif (PAM) of CRISPR nucleases is a short DNA sequence that must be recognized by the enzyme to initiate target binding³. Thus, the availability of PAMs determines what sequences can be targeted by that protein. Accurate and scalable PAM characterization is therefore important for the development and assessment of genome editing technologies. Wild-type Cas9 from Streptococcus pyogenes (WT SpCas9) requires an NGG PAM^{4,5} (where ‘N’ is any nucleotide), limiting targeting to sites bearing this sequence.

To facilitate a large-scale rational engineering approach to develop SpCas9 variants capable of targeting new PAM sequences, we required a high-throughput PAM determination assay (HT-PAMDA) that could rapidly and comprehensively profile the PAM preferences of dozens or even hundreds of SpCas9 variants. A scalable assay to fulfill these criteria would: (1) preclude protein expression and purification as it is not feasible to purify dozens or hundreds of proteins at scale (as was previously described for modest numbers of Cas12a variants⁶; or others described for a small number of variants using un-normalized lysates⁷), (2) would optimally be performed in vitro with conditions approximating a human cell context, and (3) would not be performed in bacteria or bacterial lysates (as we had done previously for SpCas9 and SaCas9 variants^{8,9}) due to intrinsic differences between activities in bacteria and human cells that might result from expression levels, post-translational modification, endogenous factors, etc.

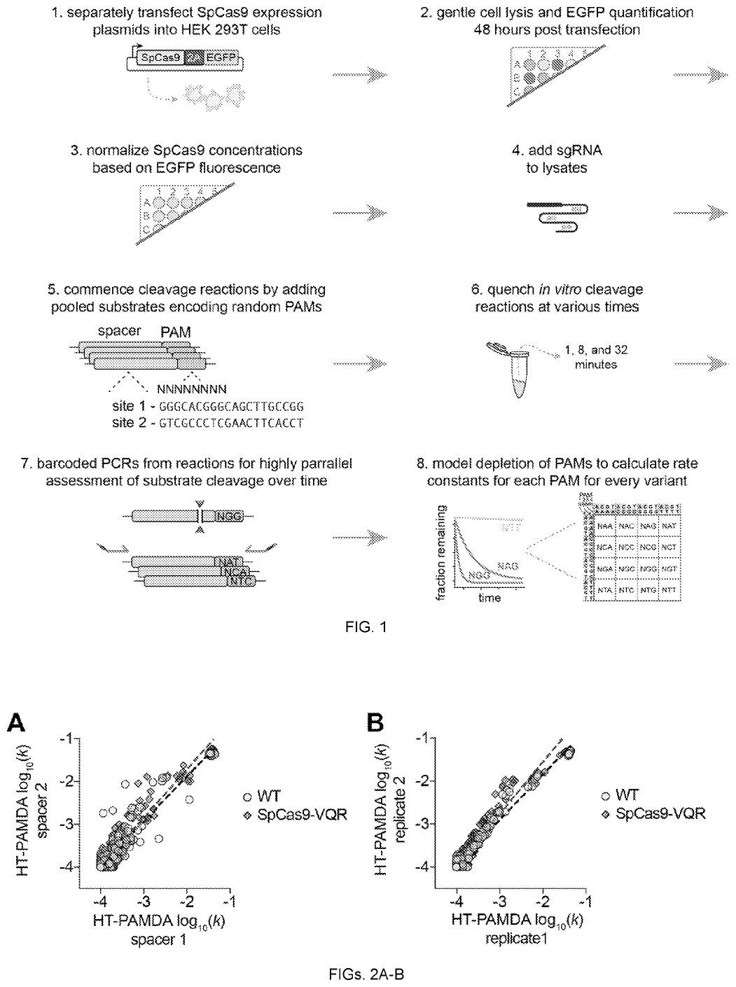

To fulfill these criteria, we developed the HT-PAMDA that first relies on the expression of SpCas9 variants in human cells, a step that can be easily arrayed and thus performed in high-throughput (FIG. 1). The variable expression of SpCas9 proteins across different transfections is measurably linked to the expression of a 2A-EGFP fluorescence, permitting the normalization of SpCas9 protein concentrations by using a defined amount of EGFP based on a fluorescein standard curve. A constant amount of SpCas9 human cell lysate is then subject to a time-course in vitro cleavage reaction of two separate libraries harboring distinct spacer sequences and 8 nucleotide randomized PAM sequences (FIG. 1). Targeted sequencing of the libraries at various time points allows quantitation of the rate of depletion of each PAM from the population over time via modeling the depletion as exponential decay; the rate constant of depletion for each PAM therefore enables us to calculate comprehensive PAM preferences for each SpCas9 variant.

Optimization and Validation of HT-PAMDA

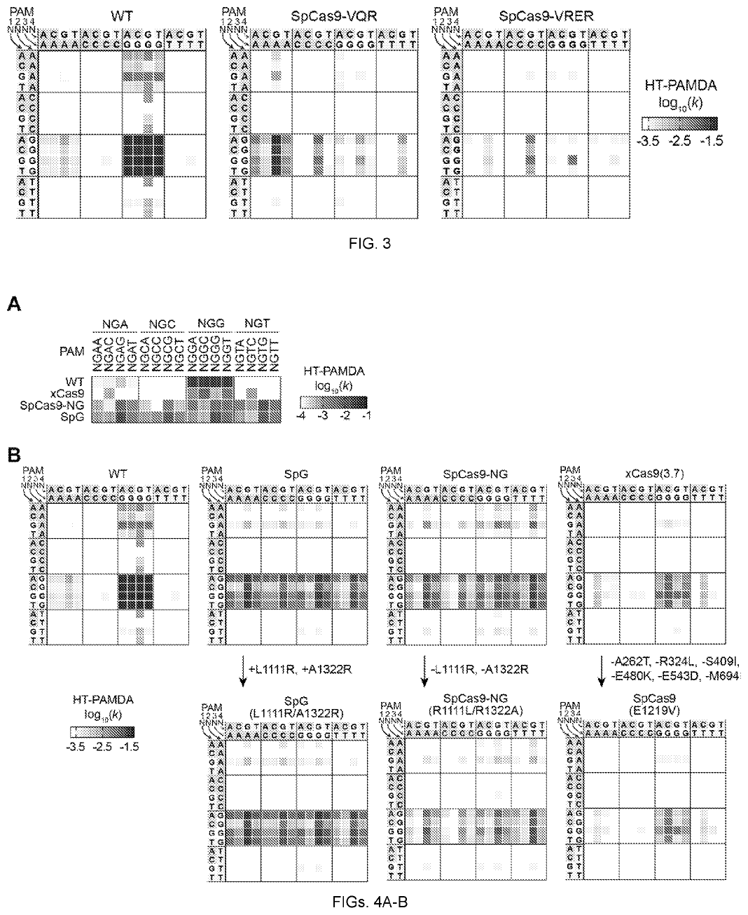

In general, we found that the HT-PAMDA profiles for WT SpCas9 and SpCas9-VQR (a variant that we previously engineered to target sites with NGA PAMs⁹) were highly reproducible across two different spacer sequences (FIG. 2A) and across technical replicates (FIG. 2B). Furthermore, the complete NNNN HT-PAMDA profiles of WT SpCas9, SpCas9-VQR, and SpCas9-VRER (a variant that we previously engineered to target sites with NGCG PAMs⁹) were consistent with their previously described NGG, NGA, and NGCG preferences, respectively, established using alternate methods⁹ (FIG. 3). We also characterized the NGNN or complete NNNN PAM profiles of xCas9(3.7), SpCas9-NG, and a new SpG variant (FIGS. 4A and 4B, respectively). With WT SpCas9 we observed targeting of NGG>NAG>NGA PAMs (consistent with prior reports^{4,9}), xCas9 demonstrated weaker targeting of NGG and NGNC, SpCas9-NG could target NGN, and SpG exhibited the most robust targeting of NGN (FIG. 4B). Interestingly, with SpCas9-NG and SpG we observed a minor ability to target sites with a subset of the NANN PAMs (especially GANN PAMs). These results demonstrate that HT-PAMDA recapitulates known PAM preferences and can in principle be scaled to large numbers of SpCas9 variants.

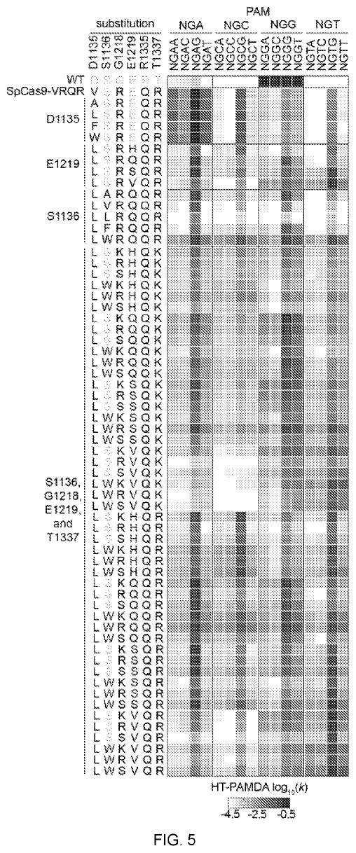

While attempting to engineer an SpCas9 variant capable of more relaxed targeting, we utilized HT-PAMDA to sequentially determine the contributions of dozens of substitutions at six critical positions in the PAM-interacting domain of SpCas9 (D1135, S1136, G1218, E1219, R1335, and T1337) (FIG. 5). The use of HT-PAMDA allowed us to identify several new SpCas9 variants bearing combinations of substitutions at these six important residues that exhibited more balanced tolerances for any nucleotide at the 3^{rd} and 4^{th} PAM positions (FIG. 5). One variant bearing D1135L/S1136W/G1218K/E1219Q/R1335Q/T1337R substitutions, referred to herein as SpG, exhibited the most even targeting of NGA, NGC, NGG, and NGT PAMs.

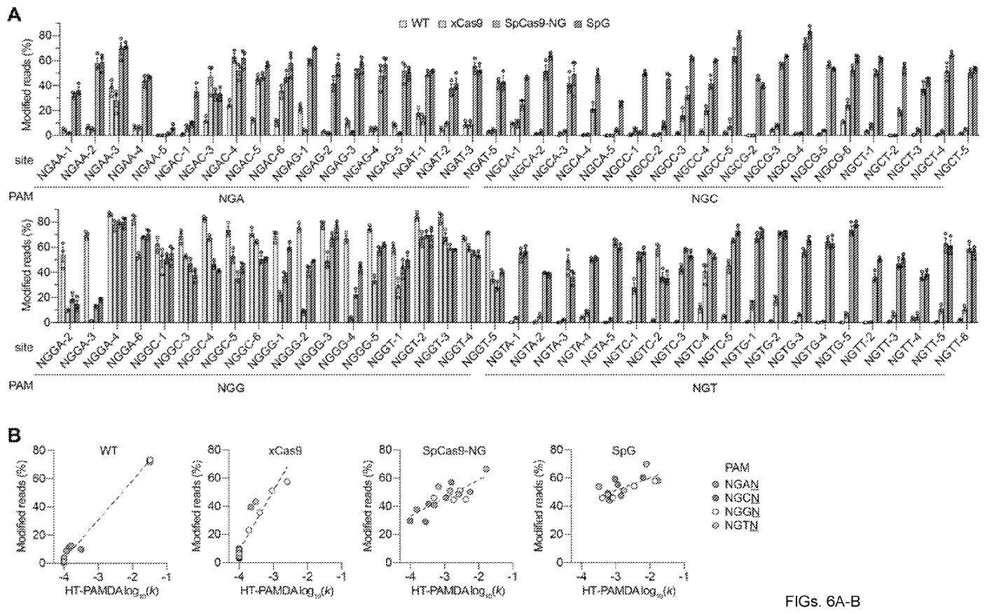

Next we sought to determine whether the HT-PAMDA results accurately recapitulated the PAM preferences of Cas9 enzymes in human cells. To do so, we performed a large number of gene editing experiments in human cells across target sites bearing NGNN PAMs with WT SpCas9, xCas9, SpCas9-NG, and SpG (FIG. 6A). In general, we found a good correlation between the mean activities on each PAM that we observed in human cells compared to the PAM preferences as determined by HT-PAMDA (FIG. 6B).

### Example 2. Optimization and Validation of Cytosine Base Editor PAM Characterization Assay

Base editor (BE) proteins are fusions of catalytically attenuated Cas9 variants to deaminase domains to mediate specific nucleotide changes in human cells^{1,2,11}. The PAM requirements of BEs have generally been assumed to be consistent with the PAM requirements of CRISPR nucleases, yet it remains to be comprehensively determined whether that they exhibit distinctive preferences. To assess whether SpCas9 nucleases and BEs exhibit consistent PAM profiles, we adapted the HT-PAMDA assay to function in the absence of SpCas9-mediated DNA cleavage. The PAM profiles generated by HT-PAMDA are dependent on the depletion of library members over time due to plasmid cleavage, yet base editors do not intentionally cleave DNA (rather, DNA binding events are followed by nicking and deamination).

Cytosine base editors (CBEs) enable the generation of C-to-T mutations in human cells¹. To determine the PAM profiles of CBEs, we adapted HT-PAMDA to develop a cytosine base editor high-throughput PAM determination assay (CBE-HT-PAMDA; FIG. 7). CBE-HT-PAMDA is similar to HT-PAMDA, but instead of double-strand DNA cleavage by SpCas9, it relies on SpCas9-based nicking and deamination of a cytosine by the tethered rAPOBEC1 domain. The combination of a target strand nick and a non-target strand deamination event is later converted to a double strand break using USER enzyme to remove the uracil base and cleave the non-target strand backbone, depleting CBE-targetable PAM-containing substrates from the library (FIG. 7).

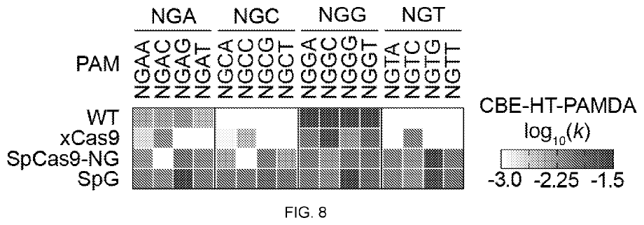

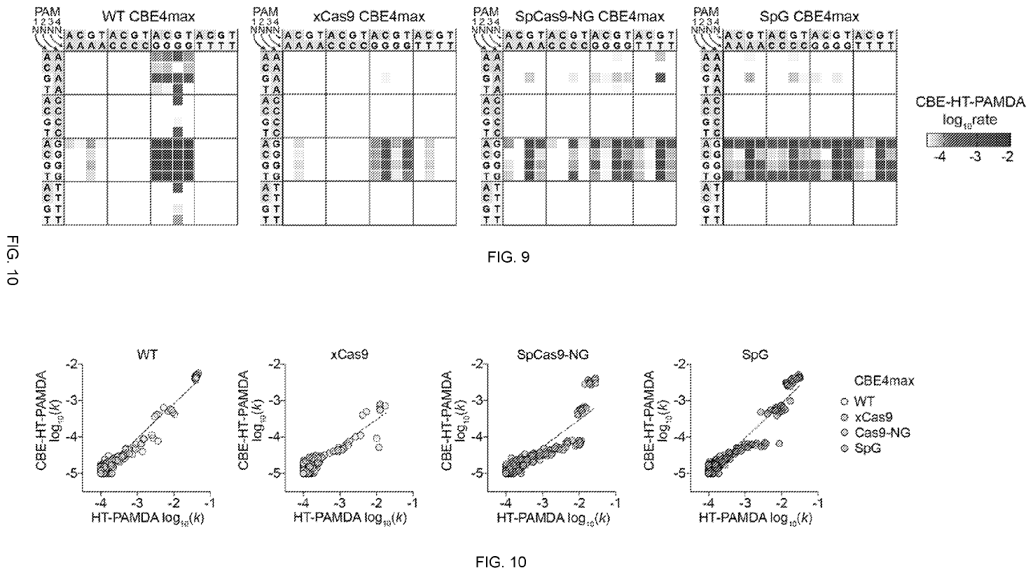

Compared to HT PAMDA for nucleases (FIG. 4), with CBE-PAMDA-HT we observed similar CBE PAM profiles for WT-SpCas9, xCas9, SpCas9-NG, and SpG (NGNN profiles, FIG. 8; complete NNNN profiles, FIG. 9). As we found for the nucleases, the WT-CBE could target NGG>NAG>NGA, xCas9 exhibited more weak targeting of NGG and NGNC, SpCas9-NG could target NGN, and SpG exhibited the most robust targeting of NGN. We also observed reasonable agreement the between HT-PAMDA and CBE-HT-PAMDA log₁₀ rates for the PAMs of the same four variants (FIG. 10). Thus, we conclude that nuclease and CBE versions of different SpCas9 variants exhibit comparable PAM profiles.

### Example 3. Optimization and Validation of an Adenine Base Editor PAM Characterization Assay

Adenine base editors (ABEs) enable the generation of A-to-G mutations in human cells². To characterize the PAM preferences of ABEs, we developed an adenine base editor high-throughput PAM determination assay (ABE-HT-PAMDA; FIG. 11). Rather than relying on cleavage of both DNA strands by SpCas9 to deplete sequences as in HT-PAMDA, ABE-HT-PAMDA relies on SpCas9 nicking of the target strand and deamination of an adenine to inosine in the non-target strand by the TadA domains of the ABE². During the in vitro ABE-HT-PAMDA protocol, the combination of a target strand nick and a non-target strand deamination event is later converted to a double strand break using Endonuclease V (NEB) to nick the non-target strand at the second phosphodiester bond 3′ of the inosine (FIG. 11).

Compared to HT PAMDA for nucleases (FIG. 4), with ABE-PAMDA-HT we observed similar ABE PAM profiles for WT-SpCas9, xCas9, SpCas9-NG, and SpG (FIG. 12). With WT-ABE we observed targeting of NGG>NAG>NGA PAMs, xCas9 demonstrated weaker targeting of NGG and NGNC, SpCas9-NG could target NGN, and SpG exhibited the most robust targeting of NGN (FIG. 12). Once again, with SpCas9-NG and SpG we observed a minor ability to target sites with a subset of the GANN PAMs.

### Example 4. Development of a Single Nucleotide Specificity Characterization Assay for Nucleases

Beyond their PAM requirements, there are other important properties of CRISPR nucleases that must be understood. It has been thoroughly established that SpCas9 and other CRISPR nucleases exhibit off-target effects because the enzymes tolerate substitutions in their binding sites¹²⁻¹⁵, so it is imperative to determine their tolerance to bind to or cleave off-target sites. In previous work we engineered high-fidelity SpCas9 and AsCas12a variants that have improved genome-wide specificity profiles^{6,10,16}. However, these and other enzymes still remain unable to discriminate against DNA targets that bear single mismatches compared to the intended on-target site. It is therefore important to have assays that enable understanding of these parameters which are critical for the safe use of enzymes, and also required for improving their specificities.

We therefore sought to develop assays that would enable the rapid profiling of the tolerance of Cas9 and Cas12a enzymes to single nucleotide substitutions in their target sites. To do so, we developed an assay that was technically similar to the PAMDA but instead of establishing PAM preferences, would enable thorough characterization of single mismatch tolerance. Thus, in place of using a library of substrates encoding random PAM sequences, we designed and constructed a spacer mismatch depletion assay (SPAMDA) library containing a perfectly matched substrate, those bearing all possible single substitutions across a 39 nt sequence, and 10 controls bearing multiple substitutions, insertions, or deletions (see FIG. 13 panel 3 and Methods). Each substrate of the library also encodes a unique 8 nt barcode to enable identification of each substrate irrespective of sequencing errors (that might generate erroneous single nt mismatch calls). This library of plasmids could then be used as a substrate for in vitro cleavage reactions with purified Cas9, Cas12a, or other CRISPR proteins. Additionally, the library is designed with multiple PAM sequences of common CRISPR enzymes (NGG (3′) for SpCas9, NNGRRT (3′) for SaCas9, and TTTV (5′) for Cas12a orthologs) falling within in the 39 nt sequence to enable characterization of multiple nucleases, each with multiple spacer sequences, all with a single library (FIG. 13). In SPAMDA, a constant amount of the normalized purified protein is utilized in time-course in vitro cleavage reactions of the libraries. Targeted sequencing of the cleavage reactions at various time points allows quantitation of the rate of depletion of each spacer substrate from the population over time; the rate constant for each matched or mismatched substrate therefore enables us to determine a comprehensive single nt specificity profile for each Cas9 or Cas12a variant.

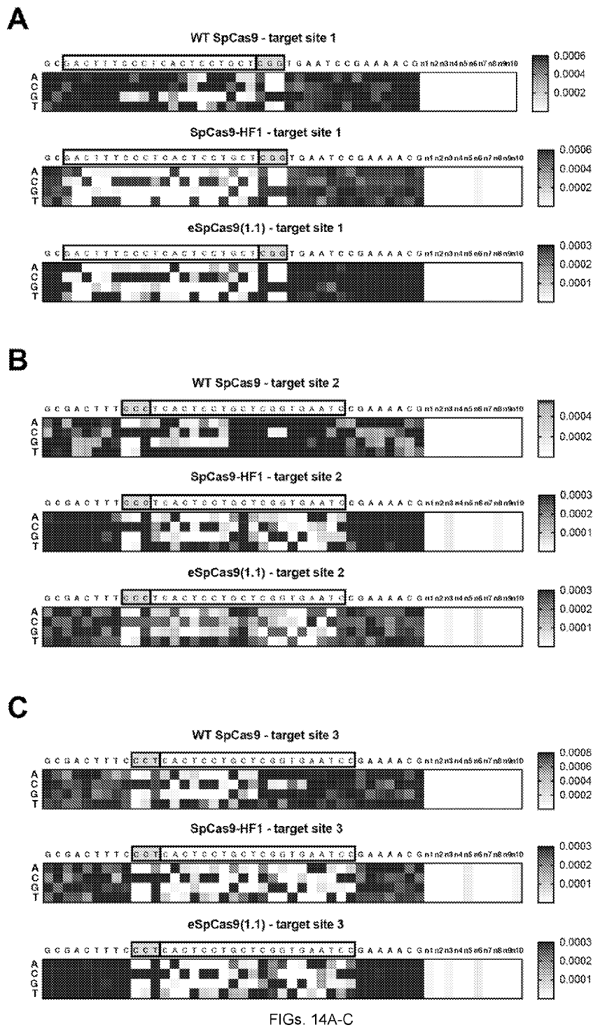

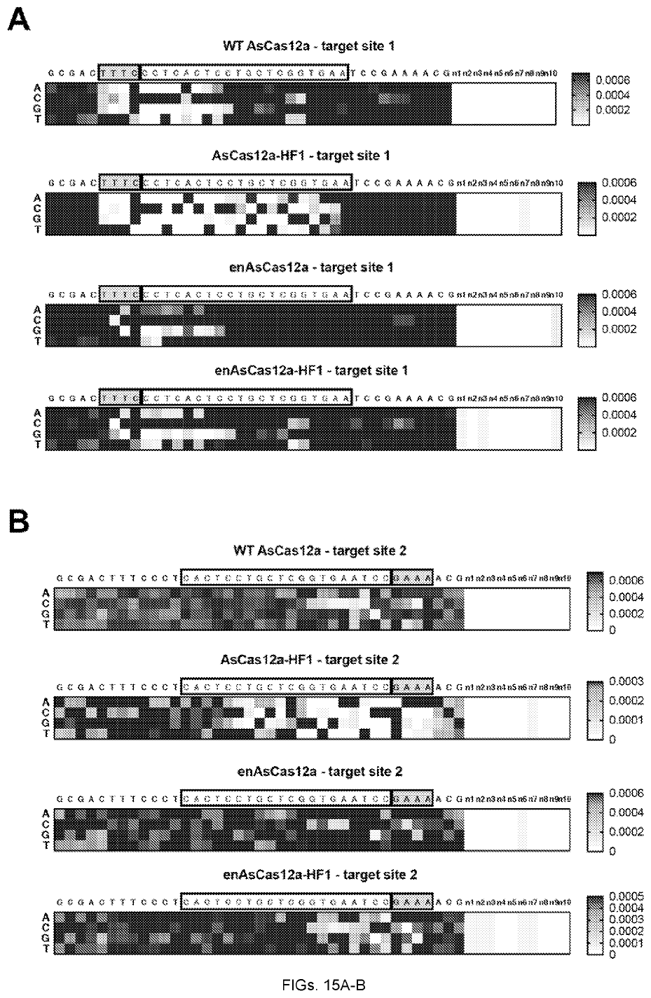

To optimize and validate the SPAMDA assay, we purified WT SpCas9, SpCas9-HF1 (bearing N497A/R661A/Q695A/Q926A substitutions)¹⁰, and eSpCas9(1.1) (bearing K848A/K1003A/R1060A substitutions)¹⁷. While both SpCas9-HF1 and eSpCas9(1.1) were previously shown to exhibit dramatically improved genome-wide specificities (against off-target sites with 2+mismatches) using GUIDE-seq¹² or other methods, they were both still able to cleave off-target sites bearing single mismatches¹⁶. In our experiments against 3 different target sites encoded in the same SPAMDA library (FIGS. 14A-14C), we observed that WT SpCas9 stringently specified an NGG PAM, was highly tolerant of PAM-distal single nt substitutions, and was mostly intolerant of PAM-proximal single nt substitutions. These features are consistent with prior reports that established these properties using lower-throughput methods^{16,18}. Across these same 3 target sites, we then examined the tolerances of SpCas9-HF1 and eSpCas9(1.1) to single mismatches using SPAMDA. We observed major improvements in single nucleotide intolerance compared to WT SpCas9, with SpCas9-HF1 exhibiting the greatest rejection of target sites bearing single substitutions in all parts of the target sites (FIGS. 14A-14C).

We then wondered whether we could use the same SPAMDA library to characterize the single nucleotide specificities of other CRISPR nucleases, including those from the Cas12a family¹⁹. We and others have previously shown that WT AsCas12a generally has high genome-wide specificity against target sites bearing 2+mismatches^{13,20}, but can exhibit a more relaxed tolerance of substitutions in the PAM and across certain positions of the spacer^{6,13}. In addition to WT AsCas12a, we also purified AsCas12a-HF1 (bearing an N282A substitution and previously shown to improve specificity), enAsCas12a (bearing E174R/S542R/K548R substitutions and previously shown to exhibit ˜7-fold relaxed recognition of new PAM sequences along with ˜2-3-fold improved on-target activity), and enAsCas12a-HF1 (bearing E174R/N282A/S542R/K548R substitutions, a high-fidelity version of enAsCas12a)⁶. SPAMDA characterization of these four AsCas12a variants across two target sites using the same SPAMDA library largely recapitulated (FIGS. 15A and 15B) the known preferences and tolerances of these enzymes. Importantly, both high-fidelity proteins exhibited reduced targeting of substrates bearing single mismatches when compared to their wild-type or enAsCas12a counterparts.

Collectively, these results show that SPAMDA can rapidly recapitulate known properties of naturally occurring and engineered CRISPR-Cas9 and -Cas12a enzymes.

### Example 5. Development of a High-Throughput Specificity Characterization Assay for Nucleases

Having established that SPAMDA can accurately determine the single nucleotide preferences of several different CRISPR proteins, we then wondered whether we could optimize a high-throughput version of SPAMDA (HT-SPAMDA) to improve scalability (FIG. 16). To do so, we utilized the same SPAMDA library bearing all single mismatches across a 39 nt sequence, but instead of using purified protein we utilized human cell lysates containing expressed CRISPR proteins (as done for the HT-PAMDA assays). The variable expression of Cas9 or Cas12a proteins across different transfections is linked to the expression of a 2A-EGFP fluorescence, permitting the normalization of nuclease concentrations based on a fluorescein standard curve (FIG. 16). Then, a constant amount of the normalized human cell lysates are then subject to time-course in vitro cleavage reactions of the SPAMDA libraries, with quantification of matched or mismatched substrate depletion enabling us to determine a comprehensive single nt specificity profile for each variant.

To validate the HT-SPAMDA, we utilized WT AsCas12a protein normalized from human cell lysates for in vitro cleavage reactions of two sets of target sites encoded within the SPAMDA library (FIG. 17). Similar to the results using purified protein for SPAMDA, with HT-SPAMDA and WT AsCas12a we observed a general preference for the canonical TTTV PAM sequences (where V is any nucleotide but T) with a minor tolerance for C substitutions. We also observed fairly robust intolerance of single nt substitutions in the PAM proximal region of the spacer, with high tolerance for single substitutions across the remainder of the spacer sequence (FIG. 17).

Together, these results demonstrate that it is feasible to utilize normalized human cell lysates in the HT-SPAMDA assay to comprehensively determine the single mismatch profile of CRISPR nucleases. The HT-SPAMDA assay should be extensible to other CRISPR proteins, including different Cas9 and Cas12a orthologs, CBEs, ABEs, and others.

### Example 6. Scalable Characterization of the PAM Requirements of CRISPR-Cas Enzymes using HT-PAMDA

This example describes an exemplary detailed protocol for a high-throughput PAM determination assay (HT-PAMDA) method that enables scalable characterization of the PAM preferences of different Cas proteins. Here, we provide a step-by-step protocol for the method, discuss experimental design considerations, and highlight how the method can be used to profile naturally occurring CRISPR-Cas9 enzymes, engineered derivatives with improved properties, orthologs of different classes (e.g. Cas12a), and even different platforms (e.g. base editors). A distinguishing feature of HT-PAMDA is that the enzymes are expressed in a cell type or organism of interest (e.g. mammalian cells), permitting scalable characterization and comparison of hundreds of enzymes in a relevant setting unlike previously available assays. HT-PAMDA does not require specialized equipment or expertise and is cost-effective for multiplexed characterization of many enzymes. The protocol enables comprehensive PAM characterization of dozens or hundreds of Cas enzymes in parallel in less than two weeks.

Overview of the Workflow

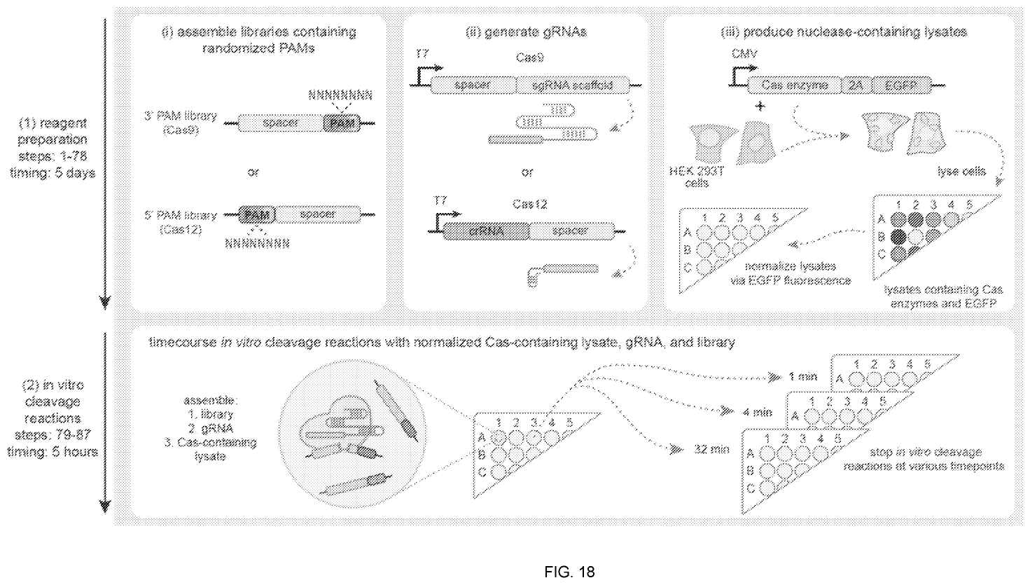

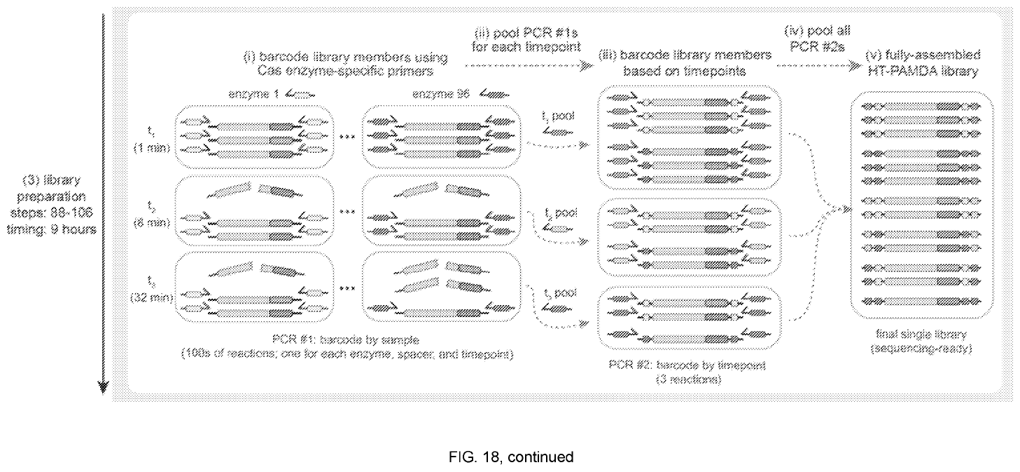

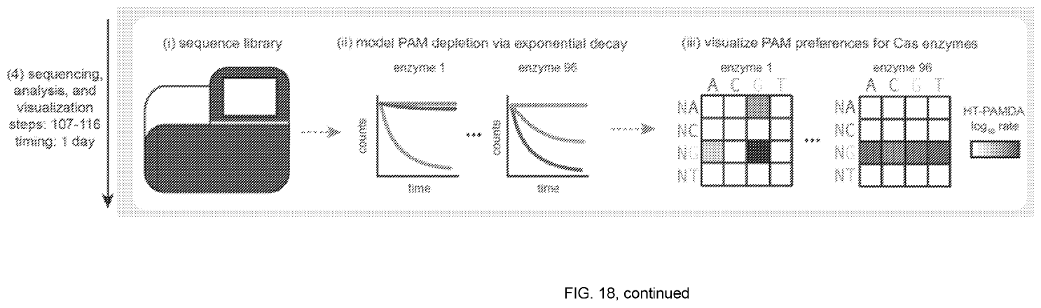

HT-PAMDA consists of four major steps (FIG. 18): (i) reagent preparation (cloning the randomized PAM library, gRNA preparation, and production of nuclease-containing lysate), (ii) in vitro cleavage reactions, (iv) library preparation, and (iv) sequencing, analysis, and visualization.

Randomized PAM library (substrate library) cloning (Steps 1-28)

The randomized PAM libraries, or substrate libraries, are the substrates to be used in the in vitro cleavage reactions. These libraries have two critical features: (i) a fixed spacer sequence, and (ii) a region of randomized nucleotides in place of the PAM (FIG. 18). Appropriate design of both features is important for accurate PAM characterization.
(i) The spacer. Libraries should be constructed with spacer sequences known to be efficiently targeted. Constructing multiple libraries with distinct spacer sequences enables potential spacer-specific effects on PAM preference to be accounted for and performing the assay on a second library also serves as a technical replicate, as the in vitro cleavage reactions are performed separately. Additional spacer design considerations apply when adapting HT-PAMDA for characterizing base editors (e.g. having targetable bases in the edit window of the target site) or other enzymes².
(ii) The PAM. To accommodate the possibility that a Cas enzyme may recognize an extended PAMs and/or can exhibit preferences beyond their core motifs, the randomized sequence should be longer than is expected to be necessary. The orientation of the randomized PAM relative to the spacer sequence is another important feature of the substrate library. The position of the PAM depends on the category of Cas enzyme being studied; generally, Cas9 nucleases require PAMs on the 3′ end of the spacer, while Cas12 nucleases require 5′ PAMs. Alternatively, libraries may be designed with spacer sequences flanking either side of the randomized PAM to generate a single substrate for Cas enzymes with either 3′ or 5′ PAM requirements.

gRNA preparation (Steps 56-65)

In HT-PAMDA, the gRNA is targeted to the spacer sequence adjacent to the randomized region of the library. There are two general approaches to preparing the gRNA: separate production of a purified gRNA (as done in the HT-PAMDA protocol) or co-transfection of the gRNA and nuclease expression plasmids into cells, combining the nuclease and gRNA production steps. The choice between these options should depend on the number of unique gRNAs to be used in the assay. If a small number of gRNAs will be used to characterize many Cas enzymes that share the same gRNA scaffold (as is the case when characterizing engineered variants of one Cas ortholog), it may be more economical to prepare the gRNA in bulk by in vitro transcription or to purchase a chemically synthesized gRNA for those that are commercially available. Alternatively, if each nuclease requires a different gRNA (for example, when characterizing multiple different Cas orthologs), it may be advantageous to co-transfect nuclease and gRNA expression plasmids into human cells when generating the lysates to avoid a large number of in vitro transcription reactions. If generating the gRNA from a lysate, the gRNA expression plasmid should be transfected in excess so that nuclease molecules are saturated with gRNA.

Production of Nuclease-Containing Lysate (Steps 66-78)

The source of Cas enzyme for HT-PAMDA from unpurified and concentration-normalized human cell lysates facilitates the scalability and accuracy of the method. To generate Cas enzymes from human cell (e.g. HEK 293T) lysates, all nuclease coding sequences should be cloned into an appropriate human expression vector that also includes a transcriptionally coupled fusion to a reporter gene to enable lysate normalization (e.g. to a 2A peptide and a fluorescent protein; FIG. 18). While obtaining sufficient quantities of Cas enzyme and reporter protein for accurate fluorescence quantification and appropriate in vitro cleavage reaction conditions is generally robust when transfecting human codon optimized constructs into HEK 293T cells, this may require optimization under different experimental conditions. Although we have not performed HT-PAMDA using Cas proteins derived from other cell types, we anticipate that Cas proteins expressed from other cells should be equivalently effective in the protocol if the cells are sufficiently transfected with the Cas expression plasmid harboring the P2A-EGFP sequence.

In Vitro Cleavage Reactions (Steps 79-87)

Time course in vitro cleavage experiments with control samples can be performed to test the functionality of both the lysate and gRNA before proceeding to a large-scale characterization. This ensures performance of reagents and is recommended to optimize conditions for new systems. In addition to the intended lysate/gRNA/PAM library combination, control samples should include (i) un-transfected lysate, (ii) nuclease-containing lysate without gRNA, and (iii) nuclease-containing lysate with non-targeting gRNA. We recommend using SpCas9 and AsCas12a as positive control nucleases for 3′ and 5′ PAM libraries, respectively. The results of these quality control experiments may be determined by NGS by following the HT-PAMDA protocol. Alternatively, for a faster quality control readout, DNA substrates resembling the PAM library but instead harboring fixed canonical and non-canonical PAMs may be used (to establish an appropriate dynamic range of in vitro cleavage rates of various substrates for the assay). Small-scale pilot experiments allow optimization of PAM library concentration, lysate concentration, and timepoint selection, where the in vitro cleavage reactions can be visualized and quantified by agarose gel or capillary electrophoresis.

It is desirable to have a control nuclease for which the performance of the nuclease in mammalian genome editing applications is known. Assay conditions should reflect the performance of the control nuclease in relevant genome editing settings. For example, with SpCas9 as a control in vitro cleavage reaction, canonical NGG PAMs should be depleted in early timepoints, and non-canonical NAG and NGA PAMs should be depleted at later timepoints to recapitulate the well-documented relative activities in human cells^{5,7,17,18,25}.

NGS library preparation and sequencing (Steps 29-48, 88-116)

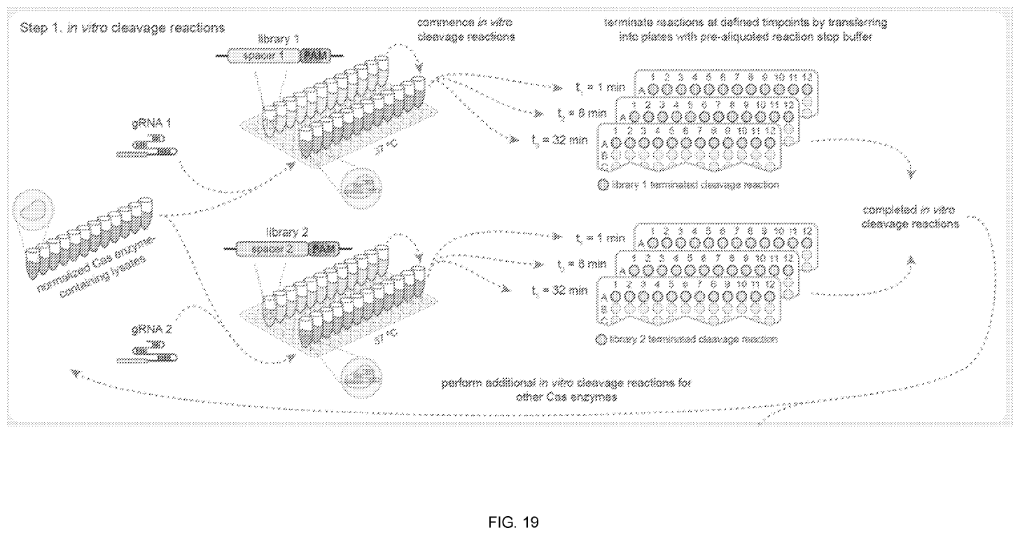

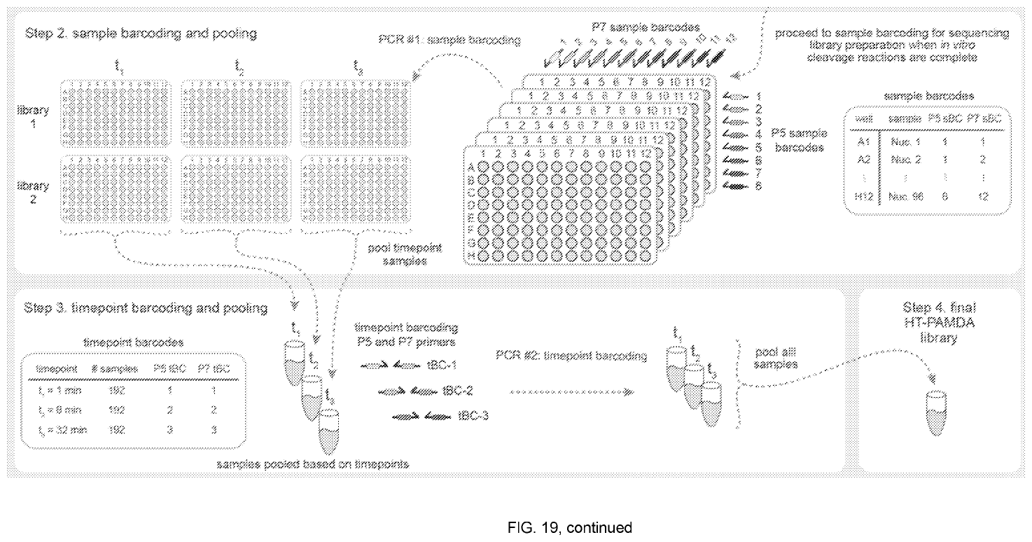

The library preparation for HT-PAMDA is designed to maximize throughput by minimizing pipetting and leveraging multiple barcoding steps (FIGS. 18 and 19). First, each reaction aliquot is labeled during PCR using primers encoding unique barcodes to index and distinguish variant nucleases. All uniquely barcoded nuclease samples from a given timepoint can then be pooled together; each timepoint pool is subsequently labeled using timepoint barcode primers (via Illumina indices) before final pooling of all samples (FIGS. 18 and 19).

The required sequencing depth per sample is dependent on the PAM representation of the substrate library, the number of nucleotides required to ascertain the complete PAM, the number of timepoints, and the number of substrate libraries. These factors considered, we recommend sequencing at a depth of approximately 750,000 reads per sample to resolve up to 5 nt of PAM preference, where a sample is comprised of one nuclease across three timepoints on two randomized PAM libraries harboring distinct spacer sequences (an average of 125,000 reads per nuclease/substrate library/timepoint). Accounting for a PhiX spike-in to increase nucleotide diversity and typical mapping rates in the analysis pipeline, there are several sequencing platforms and reagent kits that enable flexible assay throughput, including MiSeq and NextSeq.

Visualization of PAM Preference (Step 116)

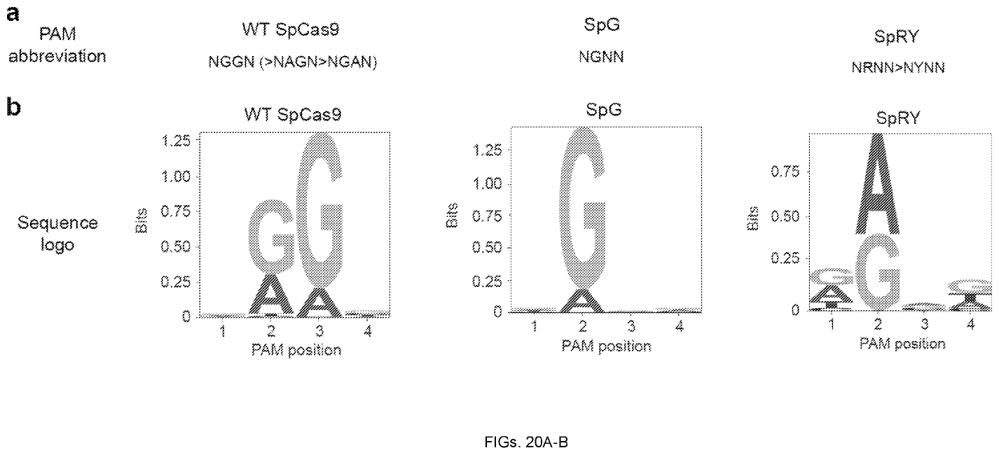

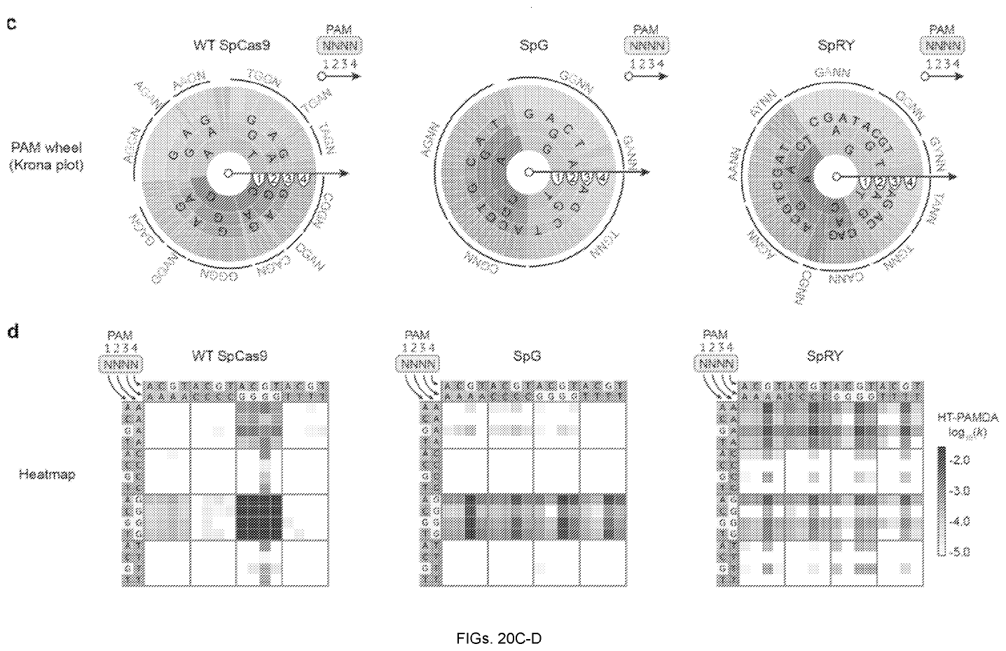

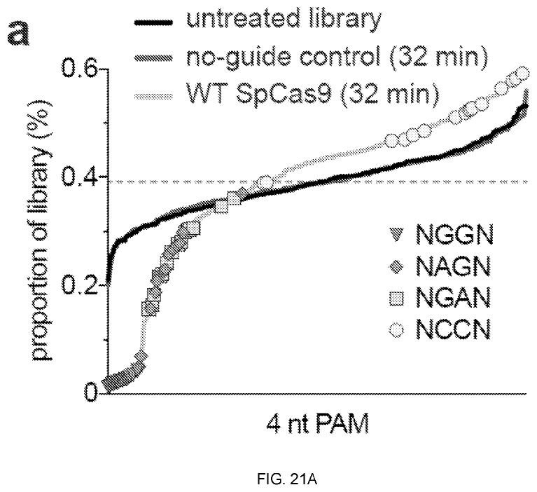

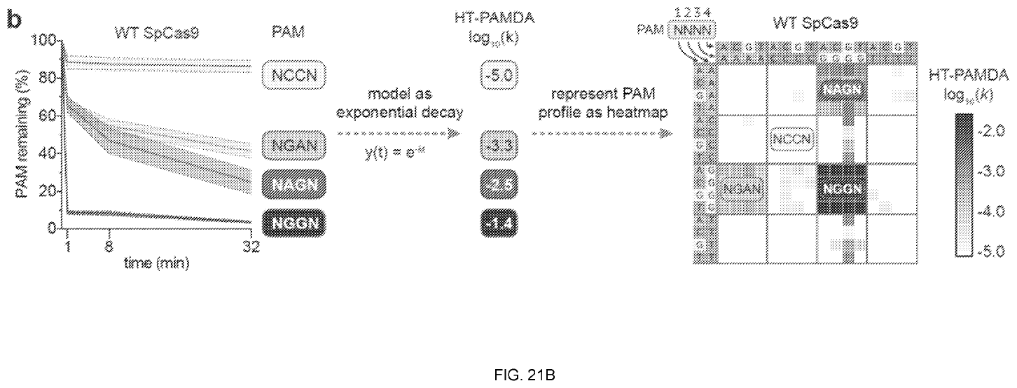

Representations of PAM preference ideally provide a comprehensive description of both PAM preference and activity. As examples, wild-type (WT) SpCas9, and the SpCas9 variants SpG (harboring the mutations D1135L/S1136W/G1218K/E1219Q/R1335Q/T1337R) and SpRY (harboring the mutations A61R/L1111R/D1135L/S1136W/G1218K/E1219Q/N1317R/A1322R/R1333P/R1335Q/T1337R), recognize NGG, NGN, and NRN>NYN PAMs, respectively (FIGS. 20a-d). Plain text abbreviations of PAM preference are convenient but minimally informative (FIG. 20a). Additionally, sequence logos have become a popular method for depicting PAM preference due to their simplicity (FIG. 20b). However, these representations treat each position of the PAM independently and provide no information about the absolute level of activity targeting any PAM. For example, with a sequence logo of the PAM of wild-type SpCas9, it can be difficult to interpret the relative differences between NRR PAMs (where R is A or G), despite their established biological ranking of NGG>NAG>NGA>>>NAA (FIG. 20b)^{17,18}. PAM wheels are a representation based on Krona plots that preserve position interdependencies (FIG. 20c)^{22,26}. However, PAM wheels indicate only PAM preference, without a measure of absolute activity. For example, PAM wheels of wild-type SpCas9 and SpG reveal that both enzymes target NGG PAMs, but do not enable a comparison of their activities (FIG. 20c). Finally, heatmap representations of PAM preference capture both position interdependencies and activity on an absolute scale (FIG. 20d), permitting representation of PAM preferences as log scale heatmaps of PAM depletion rate constants. The rate constants reflect rate of depletion for any given PAM from a library over time, and are directly comparable across nucleases to determine differences in targeting efficiency.

Beyond the choice of PAM visualization format, it's also essential to represent all bases of the PAM that influence PAM preference. Failing to do so can misleadingly represent a group of PAMs as targetable, when the group is actually comprised of both targetable and non-targetable sequences. Even thoroughly characterized nucleases have PAM preferences beyond their well-known canonical requirements so it is good practice to visualize more positions than are anticipated to influence activity. For example, while SpCas9 is known to have 2 nt of specificity for its canonical NGG PAM, the capacity to target sites with shifted NNGG PAMs is apparent when also visualizing the 4^{th} nucleotide of the PAM (FIG. 20d)^{5,18,27}.

Additional Design Considerations

Endpoint Versus Kinetics Measurements

Most experimental methods for characterizing PAM specificity are amenable to either endpoint or multiple timepoint measurements that enable calculation of kinetic parameters. While endpoint measurements are experimentally more straightforward and require less total sequencing depth, they can provide dramatically different characterizations of PAM preference depending on the selected timepoint. The use of multiple timepoints enables the determination of cleavage kinetics for each PAM, a more intrinsic metric of activity that is more informative compared to the use of a single endpoint measurement.

Alterations for base editor formats (Step 87)

While PAM depletion assays typically require DNA double-strand breaks (DSBs) to deplete targetable PAMs from the library, these assays are also adaptable for the measurement of other DNA modifications such as those made by base editors. For example, in CBE-HT-PAMDA the CBE generates target strand nicks and non-target strand C-to-U deamination events that can be converted to DSBs via treatment with USER enzyme to excise uracil nucleotides. Similarly, in ABE-HT-PAMDA, ABEs generate target strand nicks and non-target strand A-to-I deamination events that can be converted to DSBs via treatment with Endonuclease V to cleave the inosine-containing non-target strand²⁸. These assays require additional considerations, including library design to position target cytosines or adenines within the edit window of the target site, and alterations to in vitro reaction conditions to accommodate different reaction kinetics.

Assay Readout Formats by Sequencing

Most PAM determination assays can be read out by either NGS or Sanger sequencing. Sanger sequencing of PAM libraries provides a coarse description of PAM preference by averaging composition at each position of the PAM at a given endpoint. This can be rapid and affordable for a small number of samples; however, this approach occludes positional dependencies in the PAM and thus can provide an inaccurate characterization of PAM preference. NGS-based readouts provide a more complete characterization and enable sample multiplexing via barcoding that increase sample throughput while decreasing per-sample cost.

### Materials

Biological Materials
HEK 293T cells (ATCC, cat. no. CRL-3216).
XL1-Blue chemically competent E. coli (Agilent, cat. no. 200229)
XL1-Blue electrocompetent E. coli (Agilent, cat. no. 200158)

Reagents

### General Laboratory Reagents

Deoxynucleotide (dNTP) solution mix (New England BioLabs, cat. no. N0447L)
Super optimal broth (SOB) (MilliporeSigma, cat. no. H8032-500G)
D-(+)-Glucose (MilliporeSigma, cat. no. G8270-100G)
Luria-Bertani (LB) broth (MilliporeSigma, cat. no. L3022-250G)
LB agar (MilliporeSigma, cat. no. L2897-250G)
Carbenicillin disodium salt (MilliporeSigma, cat. no. C1389-1G)
Sera-Mag carboxylate-modified magnetic particles (hydrophobic) (Cytiva, cat. no. 44152105050250)
Polyethylene Glycol 8000 (PEG) (Fisher BioReagents, cat. no. BP233-100)
Sodium chloride solution (5 M) (MilliporeSigma, cat. no. 7647-14-5)
UltraPure 1M Tris-HCl, pH 8.0 (ThermoFisher, cat. no. 15568025)
Tween20 (MilliporeSigma, cat. no. P1379-100 ML)
Ethylenediaminetetraacetic acid (EDTA) solution, pH 8.0, ˜0.5 M in H2O (MilliporeSigma, cat. no. 03690-100 ML)
Ethanol solution 70% (Fisher BioReagents, cat. no. BP8201500)
Ethidium bromide solution (MilliporeSigma, cat. No. E1510-10 ML)
QX DNA Fast Analysis Kit (Qiagen, cat. no. 929008)
Purple (6×) Gel Loading Dye (New England BioLabs, cat. no. B7024S)
QIAquick Gel Extraction Kit (Qiagen, cat. no. 28704)
MinElute PCR Purification Kit (Qiagen, cat. no. 28004) Plasmids, plasmid libraries, and oligonucleotides
Plasmids required for cloning or ready-to-use plasmids and plasmid libraries (available from Addgene)
Custom oligonucleotides were used for cloning and library preparation. All oligonucleotides were ordered from Integrated DNA Technologies at the 25 nmol scale as standard desalted oligonucleotides. Higher synthesis scales might improve oligonucleotide purity. For the randomized bases of the PAM libraries, the hand-mixed base option was used.
   Substrate library construction
Klenow Fragment (3′-5′ exo-) (New England BioLabs, cat. no. M0212S)
EcoRI-HF (New England BioLabs, cat. no. R3101S)
SphI-HF (New England BioLabs, cat. no. R3182S)
SpeI-HF (New England BioLabs, cat. no. R3133S)
PvuI-HF (New England BioLabs, cat. no. R3150S)
T4 DNA ligase (New England BioLabs, cat. no. M0202S)
QIAGEN Plasmid Plus Maxi Kit (Qiagen, cat. no. 12963) gRNA preparation
RNase ZAP (Thermo Fisher Scientific, cat. no. AM9780)
QIAprep Spin Miniprep Kit (Qiagen, cat. no. 27104)
BsaI-HFv2 (New England BioLabs, cat. no. R3733S)
HindIII-HF (New England BioLabs, cat. no. R3104S)
T7 RiboMAX express large scale RNA production kit (Promega, cat. no. P1320) Tissue culture
Dulbecco's Modified Eagle's Medium (DMEM), high glucose, GlutaMAX, pyruvate (ThermoFisher, cat. no. 10569069)
PBS, pH 7.4 (ThermoFisher, cat. no. 10010031)
Fetal Bovine Serum (FBS), qualified, heat inactivated (ThermoFisher, cat. no. 10438026)
Penicillin-Streptomycin (ThermoFisher, cat. no. 15070063)
Trypsin-EDTA (0.05%), phenol red (ThermoFisher, cat. no. 25300054)

### Lysate Preparation

TransIT-X2 transfection reagent (Mirus, cat. no. MIR 6000)
Opti-MEM reduced serum medium (ThermoFisher, cat. no. 31985062)
SIGMAFAST protease inhibitor cocktail, EDTA-free (Millipore Sigma, cat. no. S8830)
HEPES buffer solution, pH 7.5 (Fisher Scientific, cat. no. NC0358126)
Sodium chloride solution (MilliporeSigma, cat. no. S6546-1 L)
Potassium chloride solution (MilliporeSigma, cat. no. 60142-500 ML-F)
Magnesium chloride solution (MilliporeSigma, cat. no. M1028-100 ML)
Glycerol (MilliporeSigma, cat. no. G5516-500 ML)
Dithiothreitol (DTT) solution (MilliporeSigma, cat. no. 646563-10X.5 ML)
Triton X-100 (MilliporeSigma, cat. no. T9284-100 ML)
Fluorescein dye (MilliporeSigma, cat. no. F2456-2.5G)
   In vitro cleavage reactions
Proteinase K (New England BioLabs, cat. no. P8107S)
   Library preparation and sequencing
QuantiFluor dsDNA system (Promega, cat. no. E2670)
Q5 High-Fidelity DNA Polymerase (New England BioLabs, cat. no. M0491 L)
Betaine solution, 5 M (MilliporeSigma, cat. no. B0300-5VL)
Exonuclease I (New England BioLabs, cat. no. M0293S)
Universal KAPA Illumina Library qPCR Quantification Kit (KAPA Biosystems, cat. no. 7960140001)
Sodium hydroxide (NaOH) solution, 2 N (Honeywell Fluka, cat. no. 352541 L)
PhiX control v3 (Illumina, cat. no. FC-110-3001)
75-cycle NextSeq 500/550 High Output v2.5 kit (Illumina, cat. no. 20024906) Equipment
Filtered sterile pipette tips
1.7 mL tubes (VWR, cat. no. 87003-294)
Axygen 96-well flat top polypropylene PCR microplate (Corning, cat. no. PCR-96-FLT-C)
Aluminum adhesive plate seal (MilliporeSigma, cat. no. Z721549-100EA)
384-well black/clear polystyrene microplates (Corning, cat. no. 3540)
8-strip tubes with cap (USA Scientific, cat. no. 1402-4708)
Axygen 25 mL disposable reagent reservoir, sterile (Corning, cat. no. RES-V-25-S)
Axygen 24-well clear V-bottom 10 mL polypropylene rectangular well deep well plate (Corning, cat. no. P-DW-10 ML-24-C)
Petri dishes (VWR, cat. no. 470210-568)
Vacuum filter flask (1 L) (MilliporeSigma, cat. no. S2HVU11 RE)
Magnetic stir bar (VWR, cat. no. 76006-402)
Breathe Easier sealing membrane for multiwell plates (MilliporeSigma, cat. no. Z763624-100EA)
Electroporation cuvettes (BTX, cat. no. 45-0124)
Tissue culture dish (150 mm) (Fisher Scientific, cat. no. 877224)
Serological pipettes (5 mL) (Fisher Scientific, cat. no. 13-678-11D)
Serological pipettes (10 mL) (Fisher Scientific, cat. no. 13-678-11E)
Serological pipettes (25 mL) (Fisher Scientific, cat. no. 13-678-11)
24-well tissue culture plates (Corning, cat. no. 3526)
INCYTO C-Chip hemocytometers (SKC Inc., cat. no. DHC-N015)
MicroAmp optical 96-well reaction plate (Applied Biosystems, cat. no. N8010560)
MicroAmp optical adhesive film (Applied Biosystems, cat. no. 4360954)
Cell culture CO₂ incubator
Magnetic stir plate
Gene Pulser Xcell Microbial System (BioRad, cat. no. 1652662)
Vortexer
Labnet mini plate spinner (Thomas Scientific, cat. no. 1225Z37)
Microcentrifuge (Eppendorf, cat. no. 5420000040)
QIAxcel Advanced Instrument (Qiagen, cat. no. 9001941)
Agarose gel electrophoresis apparatus (Fisher Scientific, cat. no. 09-528-110B)
Gel electrophoresis power source (Fisher Scientific, cat. no. FBEC300XL)
UV transilluminator (Fisher Scientific, cat. no. UV95045201)
Centrifuge (Eppendorf, cat. no. 5804)
Nanodrop spectrophotometer (ThermoFisher, cat. no. ND-2000)
Autoclave
Biological safety cabinet
Light microscope
Standard single-channel pipette set
Heated shaker-incubator for bacterial culture growth
Erlenmeyer flasks, 500 mL (Fisher Scientific, cat. no. S63273)
Serological pipettor
Multichannel pipette: 12-channel 2-20 μL
Multichannel pipette: 12-channel 20-200 μL
DynaMag-96 Side Magnet (ThermoFisher, cat. no. 12331D)
50 ml magnetic separation rack (New England BioLabs, cat. no. S1507S)
Fluorescence microplate reader (BioTek, DTX 880 Multimode Plate Reader)
96-well thermal cycler (Applied Biosystems, cat. no. A24811)
qPCR machine (Applied Biosystems, Quant Studio 3)
Illumina sequencing platform (MiSeq, NextSeq, or other) Software
bcl2fastq2 (Illumina)
Python 3 (https://www.python.org/downloads/)
HT-PAMDA (https://github.com/kleinstiverlab/HT-PAMDA)

### Reagent Setup

Solutions
Glucose solution (1 M)

Dissolve 18 g of glucose in 100 mL of water. Filter or autoclave to sterilize and store aliquots at room temperature (22° C.) or −20° C. indefinitely.
Carbenicillin stock (1000×, 100 mg/mL)

Dissolve 1 g of carbenicillin disodium salt in 10 mL of water. Mix to dissolve, aliquot, and store at −20° C. for at least one year.
Sodium chloride-tris-EDTA (STE) buffer (10×)

To make 10× STE buffer, combine 1 mL of 1 M Tris-HCl pH 8.0, 1 mL of 5 M NaCl, 200 μL of 0.5 M EDTA pH 8.0, and nuclease-free water to 10 mL (1× STE: 10 mM Tris-HCl pH 8.0, 50 mM NaCl, and 1 mM EDTA). Filter or autoclave to sterilize and store aliquots at room temperature indefinitely.
1× TE buffer (10 mM Tris-HCl, 1 mM EDTA)

Combine 5 mL 1 M Tris-HCl (pH 8.0), 1 mL 0.5M EDTA (pH 8.0), and nuclease-free water to 500 mL. To prepare 0.1× TE, diluted 1:10 using nuclease-free water. Filter or autoclave to sterilize and store aliquots at room temperature indefinitely.
SPRI buffer

Combine 135 g of PEG-8000, 150 mL of 5 M NaCl, 7.5 mL of 1 M Tris-HCl pH 8.0, 1.5 mL of 0.5 M EDTA, 375 μL of Tween20, and sterile-filtered deionized water to a final volume of 750 mL. Add a magnetic stir bar and stir on a magnetic stir plate. The solution may be heated to approximately 50° C. to facilitate dissolving the PEG. When dissolved, the solution should be completely transparent. Sterile filter the buffer and store at room temperature indefinitely. The buffer is highly viscous and will pass slowly through the filter.
Cleavage buffer (10×)

To make 10× cleavage buffer, combine 10 mL of 1 M Hepes pH 7.5, 30 mL of 5 M NaCl, 5 mL of 1 M MgCl₂, and deionized water to a final volume of 100 mL (1× cleavage buffer: 10 mM Hepes pH 7.5, 150 mM NaCl, and 5 mM MgCl₂). Filter or autoclave to sterilize and store aliquots at room temperature indefinitely.

Prior to use in in vitro cleavage reactions, a 1 mL aliquot of 10× cleavage buffer should be supplemented with 10 μL of 1 M DTT (to make 10× cleavage buffer+DTT).
Lysis buffer (1×)

To make 1× lysis buffer, combine 2 mL of 1 M Hepes pH 7.5, 10 mL of 1 M KCl, 500 μL of 1 M MgCl₂, 5 mL of glycerol, SIGMAFAST Protease Inhibitor Cocktail tablet (EDTA-Free), 100 μL of 1 M DTT, 100 μL of Triton X-100, and sterile-filtered deionized water to a final volume of 100 mL. Mix until the protease inhibitor tablet is dissolved. (1× lysis buffer: 20 mM Hepes pH 7.5, 100 mM KCl, and 5 mM MgCl₂, 5% (v/v) glycerol, 1 mM DTT, 0.1% (v/v) Triton X-100, and protease inhibitor). The lysis buffer without DTT and the protease inhibitor can be filtered or autoclave to sterilize and aliquots can be stored at room temperature indefinitely. Fully reconstituted lysis buffer should be prepared fresh.
Reaction stop buffer (1×)

For stopping in vitro cleavage reactions, prepare a solution of 1× stop buffer by combining 0.5 μL Proteinase K (20 mg/ml), 0.5 μL 500 mM EDTA (pH 8.0), and 4 μL water for each reaction to be stopped (for final concentrations of 2 mg/mL Proteinase K and 50 mM EDTA).
Fluorescein dye stock solution

Prepare a stock solution of 2.5 mM fluorescein dye. First, dissolve 1 mg of fluorescein free acid in 1 mL of 1 M NaOH. Next, dilute the 1 mg/mL dye solution to 2.5 mM in 1× cleavage buffer. Store 1 mL aliquots at −20° C. for at least one year.
NaOH (0.2 N)

Dilute 10 μL of 2 N NaOH in 90 μL of nuclease-free water.
Tris-HCl and Tween 20 solution (10 mM Tris-HCl, 0.1% Tween 20)

Combine 100 μL of 1 M Tris-HCl pH 8.0, 10 μL of Tween 20, and nuclease-free water to 10 mL. Filter or autoclave to sterilize and store aliquots at room temperature indefinitely.
Tris-HCl (200 mM)

Combine 200 μL of 1 M Tris-HCl pH 8.0 and 800 μL of nuclease-free water. Filter or autoclave to sterilize and store aliquots at room temperature indefinitely.

### Media

HEK 293T culture medium

In a biological safety cabinet, combine Dulbecco's Modified Eagle Medium (DMEM), Fetal Bovine Serum (FBS; final 10% v/v), and Penicillin-Streptomycin (100 U/mL). Sterile filter media with a vacuum flask. Media should be stored at 4° C. and warmed to 37° C. before use. Fresh media should be prepared every few months.
SOC (1 L)

Reconstitute 28 g of super optimal broth (SOB) powder with distilled water to 1 L. Dissolve powder by swirling. Autoclave at 121° C. for 30 minutes to sterilize. Let the medium cool to room temperature; once cooled, add 20 mL of sterile-filtered 1 M glucose. Prepared SOC can be stored at room temperature indefinitely if kept sterile.
LB broth (1 L)

Reconstitute 25 g of lysogeny broth (LB) powder with deionized water to 1 L. Dissolve powder by swirling. Autoclave at 121° C. for 30 minutes to sterilize. Let the medium cool to room temperature before adding antibiotic. For LB with Carbenicillin: Add 1 mL of Carbenicillin at 100 mg/mL to 1 L of LB broth. LB with Carbenicillin can be stored at 4° C. for 2 weeks. For LB with Kanamycin: Add 1 mL of Kanamycin at 50 mg/mL to 1 L of LB broth. LB with Kanamycin can be stored at 4° C. for 2 weeks.
LB agar (1 L)

Reconstitute 40 g of LB agar powder with deionized water to 1 L. Dissolve powder by swirling. Add a magnetic stir bar. Autoclave at 121° C. for 30 minutes to sterilize. After autoclaving but while the solution is still hot, stir slowly at room temperature using the magnetic stir bar and a magnetic stir plate. Let the medium cool to approximately 50° C. while stirring before adding antibiotic. For LB with carbenicillin: Add 1 mL of Carbenicillin at 100 mg/mL to 1 L of LB agar and stir for several minutes. For LB with kanamycin: Add 1 mL of Kanamycin at 50 mg/mL to 1 L of LB agar and stir for several minutes. Before the media cools and solidifies, pour approximately 20 mL of LB agar with antibiotic into 100-mm Petri dishes. Cover Petri dishes once poured and store at room temperature until the plates have cooled to room temperature. Store LB agar plates in plastic bags at 4° C. for up to a month.

SPRI Bead Preparation
Prepare SPRI beads as previously described²⁹. Briefly, prepare Sera-Mag SpeedBeads in a 50 mL conical tube using an appropriate magnetic rack. Wash the beads with 0.1× TE buffer (for a total of 5 washes using 40 mL 0.1× TE each) and then resuspend in 750 mL of SPRI buffer. Mix the solution well, aliquot, and store at 4° C. for up to 6 months (longer storage can alter the DNA fragment retention of the beads). The DNA fragment retention of the SPRI bead stock may be tested by performing a cleanup of a DNA ladder at a range of SPRI beads:DNA ladder volume ratios (recommended range of 0.5:1 to 2:1).

Preparation of Barcoded PCR Primer Plate for Library Preparation
There are two sets of primer pairs each used for two separate rounds of PCR: The first set of primers consists of the sample barcoding primers, which bind on the randomized PAM library and add both sample barcodes and Illumina read 1 (P5 end) and read 2 (P7 end) sequencing primer binding sites. The second set of primers consists of the timepoint barcoding primers, which bind to the Illumina read 1 and 2 sequencing primer binding sites (from primer set 1) and append both Illumina indices (which serve as the timepoint barcodes) and P5/P7 grafting regions. Oligos for both sets should be prepared in an arrayed plate layout. For each set of oligos, there are 8× P5 (P5-1 through P5-8) and 12× P7 (P7-1 through P7-12) primers. Lyophilized oligos can be resuspended using 0.1× TE (or other appropriate buffer) to a concentration of 100 μM.

For each set, prepare an arrayed 96-plate of 5 μM each forward and reverse primers as follows: Add 90 μL of 0.1× TE buffer to each well of a 96-well PCR plate. In a separate 8-strip tube, aliquot 70 μL of each 100 μM P5 primer in order P5-1 through P5-8. Using a multichannel, aliquot 5 μL of the primers into each column of the 96-well PCR plate such that row A contains P5-1, row B contains P5-2, etc. In a separate 12-strip tube, aliquot 50 μL of each 100 μM P7 primer in order P7-1 through P7-12. Using a channel multichannel, aliquot 5 μL of the primers into each row of the 96-well PCR plate such that column 1 contains P7-1, column 2 contains P7-2, etc. Seal tightly with an aluminum adhesive plate seal, mix by gently vortexing, spin down, and store at −20° C.

### Exemplary Procedure

PAM Library, gRNA, and Lysate Preparation

Cloning the Randomized PAM Substrate Library

The following library construction steps should be performed for each PAM library. Multiple libraries can be constructed in parallel. The steps are described specifically for the construction of a library harboring a randomized 3′ PAM encoded by the primer oBK1948 (Table 1). Until analysis of the PAM representation within the library (Step 55), the steps are otherwise identical for constructing other libraries bearing different spacers or randomized PAMs on the 5′ end of the spacer (e.g. those encoded by oligos oBK1949, oBK5962, oBK5964, or user-defined oligo designs following the same cloning strategy; Table 1). The following steps include cloning of the randomized PAM libraries, however four ready-to-use libraries are available on Addgene (two spacer sequences each for 3′ and 5′ randomized PAM libraries). To skip cloning, proceed directly to NGS validation of the library (Step 29).
1. Cloning the substrate library. Digest approximately 10 μg of the entry cassette plasmid (p11-lacY-wtx1) with EcoRI-HF, SpeI-HF, and SphI-HF for 1-4 hour(s) at 37° C. using the following reaction mix:

| Reagent | Stock concentration | Volume (μL) |
|---|---|---|
| p11-lacY-wtx1 | Variable | x |
| CutSmart buffer | 10X | 4 |
| EcoRI-HF | 20,000 units/mL | 1 |
| Spel-HF | 20,000 units/mL | 1 |
| Sphl-HF | 20,000 units/mL | 1 |
| Nuclease-free water | | 33-x |
| Total | | 40 |

2. Cloning the library into a plasmid backbone other than p11-lacY-wtx1 with a higher copy origin of replication will improve yield during DNA preparations. If using a different plasmid backbone, adjust oligo design and cloning strategy accordingly. Gel purify the reaction. Run the entire digestion reaction in gel loading dye on a 1% agarose gel with 0.5 μg/mL ethidium bromide for 45 minutes at 100 V. Excise the backbone from the gel and transfer it to a 1.7 mL tube. Purify the DNA using the QIAquick Gel Extraction Kit (or equivalent) following the manufacturer's instructions. Elute the solution in 30 μL of nuclease-free water. Quantify the purified plasmid on a NanoDrop and dilute in nuclease-free water to 40 ng/μL.
3. Resuspend the oligos oBK1948 and oBK984 to 100 μM using 0.1× TE and mix them in preparation for annealing in a 0.2 mL tube as follows:

| | Stock | Volume |
|---|---|---|
| Reagent | concentration | (μL) |
| Library template oligo (e.g. oBK1948) | 100 μM | 2 |
| Bottom extension oligo (oBK984) | 100 μM | 4 |
| STE buffer | 10X | 3.4 |
| Nuclease-free water | | 24.6 |
| Total | | 34 |

4. Anneal the oligos with the following annealing program in a thermal cycler: 95° C. for 5 min, then decrease 0.1° C. per second for 70 cycles, transfer to 4° C. or ice.
5. After the annealing program completes, add the following reagents to the annealing reaction to make the extension reaction. Mix the solution and incubate the reaction at 37° C. for 30 minutes.

| | Stock | Volume |
|---|---|---|
| Reagent | concentration | (μL) |
| Annealing reaction from Step 3 | | 34 |
| Nuclease-free water | | 8 |
| NEB buffer 2 | 10x | 5 |
| dNTPs | 10 mM | 2 |
| Klenow Fragment (3′→5′ exo-) | 5,000 units/mL | 1 |
| Total | | 48 |

6. Purify the extension reaction using the MinElute PCR Purification Kit (or equivalent kit) following the manufacturer's instructions. Elute the oligo duplex in 20 μL of nuclease-free water.
7. Digest the oligo duplex with EcoRI-HF for 1-4 hour(s) at 37° C. using the following reaction mix:

| | | Volume |
|---|---|---|
| Reagent | Stock concentration | (μL) |
| Purified oligo duplex from Step 6 | | 19 |
| CutSmart buffer | 10X | 2.2 |
| EcoRI-HF | 20,000 units/mL | 1 |
| Total | | 22.2 |

8. Purify the reaction using the MinElute PCR Purification Kit following the manufacturer's instructions. Elute the oligo duplex in 20 μL of nuclease-free water.
9. Determine the concentration of the oligo duplex by nanodrop and dilute to 30 ng/μL.
10. Set up a ligation reaction as follows to ligate the oligo duplex into the EcoRI/SpeI/SphI digested p11-lacY-wtx1 backbone. Prepare the reaction in a 1.7 mL tube, mix, and then aliquot the ligation mix into each well of an 8-strip tube with 50 μL per tube. Incubate the ligation reactions at 16° C. for approximately 16 hours.

| | | Volume |
|---|---|---|
| Reagent | Stock concentration | (μL) |
| Digested p11-lacY-wtx1 from Step 2 | 40 ng/μL | 10 |
| Digested oligo duplex from Step 9 | 30 ng/μL | 2.5 |
| T4 ligase buffer | 10X | 40 |
| T4 DNA ligase | 400,000 units/mL | 20 |
| Nuclease-free water | | 327.5 |
| Total | | 400 |

11. Pool the ligation reactions and purify the solution using the MinElute PCR Purification Kit following the manufacturer's instructions. Elute the ligation in 20 μL of nuclease-free water. Purified ligation reaction(s) can be stored at −20° C. for extended periods of time.
12. Thaw 100 μL of electrocompetent XL1-Blue cells on ice and place three electroporation cuvettes on ice. Three separate electroporations of each library will be performed. Keep the electrocompetent cells on ice at all times unless otherwise noted to maximize transformation efficiency.
13. In a 24-well (10 mL per well) block, add 3 mL of SOC medium to three wells and warm the medium to 37° C.
14. On ice in three separate 1.7 mL tubes, add 5 μL of ligation from Step 11 and 33 μL of electrocompetent cells from Step 12, such that a total of 15 μL of ligation are used across the 100 μL of cells. Mix gently by stirring with the pipette tip. Handle the cells gently. Do not pipette up and down to mix.
15. Transfer the cells to the pre-chilled electroporation cuvettes from Step 12. Pipette the mixture gently into the bottom of the cuvettes. Each cuvette should contain a mixture of 5 μL of ligation reaction and 33 μL of electrocompetent cells, for a total of three cuvettes per library. Gently tap the cuvettes so that the cells sit on the bottom of the cuvettes without air bubbles.
16. Electroporate the cells in the Gene Pulser Xcell Microbial System with the following settings.

| Parameter | Setting |
|---|---|
| Voltage | 1.75 kV |
| Capacitance | 25 μF |
| Resistance | 200 Ω |

17. Immediately following electroporation, transfer the cells in the cuvettes to 3 mL of pre-warmed SOC medium from Step 13. Rapid transfer to SOC medium is critical for transformation efficiency. Electroporate cuvettes one at a time so that the cells can be transferred to SOC medium immediately.
18. Seal the 24-well block with a breathable seal and allow the cells to recover for approximately 1 hour at 37° C., shaking at 900 RPM.
19. Plate dilutions of the electrotransformation to estimate the complexity of the library. Prepare 10- and 100-fold dilutions of the recovered cells from Step 18 by mixing 10 μL of the recovered cells with 90 μL and 990 μL of SOC medium, respectively. Plate 10 μL of each dilution on a pre-warmed LB agar plate with carbenicillin and incubate the plates at 37° C. for 16 hours. Library complexity for the full 9 mL culture can be estimated from the number of colonies that grow (see Step 22)
20. After 1 hour of growth in SOC medium, pool the recovered cells for a given library and add the full 9 mL to 150 mL of LB medium with carbenicillin.
21. Grow the culture at 37° C. for approximately 12 hours.
22. After approximately 12 hours, pellet the culture by centrifugation at 2500×g for 15 minutes and discard the supernatant. The pellets can be stored at −20° C. before proceeding with the protocol.
23. Count colonies from the plated dilutions from Step 19 to estimate the library complexity. The library complexity should exceed 100,000.

| Dilution | Colonies | Library complexity |
|---|---|---|
| 10 μL of a 10-fold dilution | x | 9,000*x |
| 10 μL of a 100-fold dilution | y | 90,000*y |

24. Prepare the plasmid libraries from the cell pellets from Step 22 with the QIAGEN Plasmid Plus Maxi Kit following the manufacturer's instructions and quantify the library on a NanoDrop.
25. Linearization of the library. Linearize approximately 10 μg of the plasmid library harboring randomized PAMs by digesting for 4 hours at 37° C. with PvuI-HF. Set up the reaction in a PCR tube as follows:

| | Stock | Volume |
|---|---|---|
| Reagent | concentration | (μL) |
| randomized PAM plasmid library | Variable | x |
| (from Step 24) | | |
| CutSmart buffer | 10X | 4 |
| Pvul-HF | 20,000 units/mL | 2 |
| Nuclease-free water | | 34-x |
| Total | | 40 |

Cleavage kinetics can differ dramatically for linear and supercoiled substrates. The reaction conditions for HT-PAMDA are optimized for a linear substrate DNA. We do not recommend using the supercoiled plasmid library as the substrate for HT-PAMDA in vitro cleavage reactions.
26. Purify the reaction with SPRI beads. Add 1.5 volumes of SPRI beads to the reaction, mix by pipetting, incubate at room temperature for 5 minutes, then place the tube on a DynaMag-96 Side Magnet (or other magnetic separator for 96-well plates). Incubate for 5 minutes or until the SPRI beads collect on the side of the tube and the solution is clear. Carefully remove the solution without disturbing the SPRI beads and discard. Wash the beads twice while keeping the tube on the DynaMag-96 Side Magnet. For each wash, add 200 μL of 70% ethanol, incubate for at least 30 seconds, and discard all the ethanol, all without disturbing the SPRI beads. After the second wash, carefully remove any residual ethanol and let the sample dry for about 3 minutes. Remove the tube from the magnet and elute by adding 40 μL of nuclease-free water directly onto the SPRI beads. Pipette to mix. Return the tube to the magnet, allow the beads to separate and transfer the eluate to a new tube, carefully avoiding carrying over SPRI beads. The incubation times necessary to separate beads will depend on the magnet strength. Ensure that the solution is clear before proceeding.
   Following the wash steps, do not let the SPRI beads dry longer than about 3 minutes as excessive drying may result in a poor recovery of DNA.
27. Quantify the purified linearized substrate library by nanodrop. The purified linearized substrate library can be stored at −20° C. for extended periods of time.
28. Run approximately 100 ng of both linearized (Step 27) and circular (Step 24) plasmid on a 1% agarose gel with 0.5 μg/mL ethidium bromide and visualize the gel under UV light to confirm that the digested plasmid is completely linearized.
29. NGS validation of library. Prepare PCRs to amplify the linearized randomized PAM plasmid libraries with a pair of PCR #1 sample barcoding primers, such as oRW1491 and oRW1501. Include a no-template control PCR.

| | Stock | Volume |
|---|---|---|
| Reagent | concentration | (μL) |
| linearized randomized PAM | 5 nM | 1.5 |
| plasmid library (Step 27) | | |
| Sample barcoding primer pair | 5 μM each | 2.5 |
| (see Reagent setup) | | |
| Q5 buffer | 5X | 5 |
| dNTPs | 10 mM each | 0.5 |
| Betaine | 5M | 2.5 |
| MgCl₂ | 50 mM | 1.5 |
| Q5 polymerase | 2,000 units/mL | 0.25 |
| Nuclease-free water | | 11.25 |
| Total (per reaction) | | 25 |

30. Run the PCRs with the following program.

| Cycle number | Denature | | Anneal | Extend | |
|---|---|---|---|---|---|
| 1 | 98° C., | 2 min | | | |
| 2-31 | 98° C., | 10 s | 67° C., 10 s | 72° C., | 10 s |

| | | |
|---|---|---|
| 32 | 72° C., | 1 min |

31. Purify the reactions with SPRI beads (as described in Step 26) by adding 1.5 volumes of SPRI beads and eluting in 25 μL of nuclease-free water.
32. Confirm amplification by running the purified reactions on a capillary electrophoresis machine or an agarose gel. For example, PCR products can be analyzed using a QIAxcel Fast Analysis cartridge on the QIAxcel Advanced (Qiagen). For all samples, combine 2 μL of PCR with 8 μL of water in a 96-well PCR plate and run the plate on the QIAxcel with the DM150 program and a 10 second injection time. The sample should have a single band with a size of 206 bp.
33. Quantify the purified reactions on a NanoDrop and prepare a dilution with a concentration of approximately 0.125 ng/μL and a volume of at least 2 μL for use as template in the second PCR. The remainder of the undiluted PCR may be stored at −20° C. for up to a year.
34. Prepare PCRs with a pair of PCR #2 timepoint barcoding primers, such as OJA1933 and OJA1941. Include a no-template control.

| Reagent | Stock concentration | Volume (μL) |
|---|---|---|
| Template amplicon (from Step 33) | 0.125 ng/μL | 2 |
| Timepoint barcode primers | 5 μM each | 2 |
| Q5 buffer | 5X | 4 |
| dNTPs | 10 mM each | 0.4 |
| Q5 polymerase | 2,000 units/mL | 0.2 |
| Nuclease-free water | | 11.4 |
| Total (per reaction) | | 20 |

35. Run the PCRs with the following program.

| Cycle number | Denature | Anneal | Extend |
|---|---|---|---|
| 1 | 98° C., 2 min | | |
| 2-11 | 98° C., 10 s | 65° C., 30 s | 72° C., 30 s |
| 12 | | | 72° C., 5 min |

36. Purify the reactions with SPRI beads as described in Step 26, adding 1.5 volumes of SPRI beads and eluting in 25 μL of nuclease-free water.
37. Confirm amplification by running the purified reactions on a capillary electrophoresis machine (as described in Step 32) or an agarose gel. The sample should have a single band with a size of 279 bp.
38. Thaw reagents for the Universal KAPA Illumina Library qPCR Quantification Kit (or equivalent) to quantify the library. Rox dye is light sensitive. Do not leave the reagent exposed to light for extended periods of time. While alternative methods of quantification are acceptable, accurate quantification is essential for determining the appropriate loading concentration for sequencing.
39. Generate 10-5 dilutions of each purified PCR from Step 36 by serial 10-fold dilutions in 1× TE buffer. Conduct serial dilutions by diluting 10 μL of library with 90 μL of TE buffer and mixing well. The remainder of the undiluted PCR may be stored at −20° C. for up to a year. Library quantification is dependent on accurate dilution of the pools.
40. Add 1 mL of 10× Illumina Primer Premix to the KAPA PCR master mix and mix (both provided in the Universal KAPA Illumina Library qPCR Quantification Kit).
41. Prepare the following qPCR master solution with enough reagent for triplicate reactions for each experimental sample and standard (6 standards), plus a no-template control. Assemble the reaction on ice.

| Reagent | Stock concentration | Volume (μL) |
|---|---|---|
| KAPA master mix | 2X | 6 |
| (primer added) | | |
| Nuclease-free water | | 1.8 |
| Rox low | 50X | 0.2 |
| Total (per reaction) | | 8 |

42. In a MicroAmp Optical 96-Well Reaction Plate, aliquot 8 μL of the qPCR master solution into each well as needed.
43. Add 2 μL of template to each well. Perform qPCRs for all experimental samples and standards in triplicate. For samples, use 10-5 dilutions of the purified PCR prepared in Step 39. For standards, use the standards as provided in the qPCR kit, with the concentrations shown below. For a no-template control reaction, add 2 μL of nuclease free water.

| Standard | Concentration (pM) |
|---|---|
| 1 | 20 |
| 2 | 2 |
| 3 | 0.2 |
| 4 | 0.02 |
| 5 | 0.002 |
| 6 | 0.0002 |

44. Seal the plate tightly with a MicroAmp Optical Adhesive Film, vortex gently, and spin down to ensure that the mixture is at the bottom of the well.
45. Run the following program on an Applied Biosystems QuantStudio 3 qPCR machine (adjusting the settings as required for quantification from a standard curve, rox dye, etc.).

| Cycle number | Denature | Anneal/ Extend |
|---|---|---|
| 1 | 95° C., 5 min | |
| 2-36 | 95° C., 30 s | 60° C., 45 s |

46. Interpreting qPCR results. Create a standard curve with the 6 triplicate standards. Determine the linear relationship between log₁₀ (concentration) and cycle threshold value by linear regression. Use this linear relationship to calculate the concentration of the pools by averaging the triplicates. Accurate quantification is important for ensuring appropriate cluster density during sequencing. For samples and standards, if one replicate is inconsistent with the other two, discard the inconsistent replicate. If no two replicates are in close agreement, repeat the qPCR. If the negative control has a signal that would meaningfully alter the quantification, repeat the qPCR.
47. Prepare an equimolar pool of the PCR from Step 36 based on the qPCR quantification.
48. Sequence the pool prepared in Step 47 on an Illumina MiSeq or NextSeq. Follow Illumina's library dilution and denaturation protocols. Sequence with 8 cycles for index 1, 8 cycles for index 2, at least 65 cycles for read 1, and at least 10 cycles for read 2. HT-PAMDA libraries have low nucleotide diversity. We recommend including 20% PhiX on the MiSeq or 37.5% PhiX on the NextSeq to increase the nucleotide diversity and improve cluster registration.
49. Library analysis. Install bcl2fastq2, available online. bcl2fastq2 runs on Linux distributions.
50. Prepare the sample sheet by entering the appropriate barcodes from the corresponding timepoint barcode primers that were used. For example, if the primers OJA1933 and OJA1941 were used, the sample sheet should contain the following values:

| Sample_ID | l7_Index_ID | index | l5_Index_ID | index2 |
|---|---|---|---|---|
| sample_id | 701 | ATTACTCG | 501 | AGGCTATA |

The P5 index (index 2) should be provided as indicated or MiSeq systems or as the reverse complement for NextSeq systems.
51. Place the sample sheet CSV in the run folder. The sample sheet must be named “SampleSheet.csv”.
52. Convert bcl files (in the run folder) to fastq files Check the output directory to ensure fastq generation was successful.
53. Download and install appropriate analysis software.
54. Launch the software
55. Enter the required inputs and run library quality control analysis.
   In Vitro Transcription of gRNAs

The steps to produce the SpCas9 gRNA targeted to spacer 1 by in vitro transcription are described below. This procedure should be carried out for each gRNA to be used in HT-PAMDA and multiple gRNAs can be produced in parallel. Custom gRNAs can be cloned into pT7-gRNA entry vectors for SpCas9 and AsCas12a, by digesting the vectors with the appropriate type IS restriction enzyme and ligating in annealed complementary oligos encoding the desired spacer sequence with the appropriate restriction site overhangs (Table 1). Entry vectors for other Cas ortholog gRNAs can be prepared with standard molecular cloning techniques. Ready-to-use T7 transcription plasmids are available on Addgene for two spacer sequences each for SpCas9 gRNAs and AsCas12a crRNAs corresponding to the substrate libraries. To avoid cloning steps, gRNAs may also be produced by in vitro transcription from oligo templates composed of a T7 promoter and the gRNA. Oligo templates can be used to produce SpCas9 sgRNAs, separate SpCas9 tracrRNA and crRNAs, AsCas12a crRNAs, and other gRNA designs. When available from commercial vendors, chemically synthesized gRNAs may also be used.

TABLE 1
Oligonucleotides.

| Oligo- | | |
|---|---|---|
| nucleotide ID | oligonucleotide description | oligonucleotide sequence* |
| oBK984 | reverse primer to fill in the bottom strand | /5Phos/CCTCGTGACCTGCGC |
| | of top strand library oligos | (SEQ ID NO: 1) |
| oBK1948 | top strand library oligo for 3′ PAM library- | GCAGgaattcGGGAGGGGCACGGGCAGCT |
| | spacer 1 with 8xN 3' PAM | TGCCGGNNNNNNNNCTNNNGCGCAGGTCA |
| | | CGAGGCATG (SEQ ID NO: 2) |
| oBK1949 | top strand library oligo for 3′ PAM library- | GCAGgaattcGGAGGGTCGCCCTCGAACT |
| | spacer 2 with 8xN 3' PAM | TCACCTNNNNNNNNCTNNNGCGCAGGTCA |
| | | CGAGGCATG (SEQ ID NO: 3) |
| oBK5962 | top strand library oligo for 5′ PAM library- | AGACCGGAATTCNNNGTNNNNNNNNNNGG |
| | spacer 3 with 10xN 5' PAM | AATCCCTTCTGCAGCACCTGGGCGCAGGT |
| | | CACGAGGCATG (SEQ ID NO: 4) |
| oBK5964 | top strand library oligo for 5′ PAM library- | AGACCGGAATTCNNNGTNNNNNNNNNNCT |
| | spacer 4 with 10xN 5' PAM | GATGGTCCATGTCTGTTACTCGCGCAGGT |
| | | CACGAGGCATG (SEQ ID NO: 5) |

*Features of oligonucleotides are indicated as follows. ‘N’: any base (randomized nucleotide).
‘X’: nucleotide of the researcher's choice (for design of custom spacer sequences). Lowercase
bases: restriction enzyme site or restriction enzyme overhangs. Underlined bases: sequence of
interest (either a spacer sequence or a primer barcode).

56. Digest approximately 5 μg of the pT7-gRNA plasmid with HindIII-HF as follows at 37° C. for 1-4 hour(s). This step linearizes the plasmid for run-off transcription.

| Reagent | Stock concentration | Volume (μL) |
|---|---|---|
| pT7-gRNA plasmid | Variable | x |
| CutSmart buffer | 10X | 4 |
| HindIII-HF | 20,000 units/mL | 1 |
| Nuclease-free water | | 35-x |
| Total | | 40 |

57. Use RNase ZAP to clean the workspace and pipettes prior to purification of the plasmid. RNase contamination will result in a low RNA yield from the in vitro transcription reaction.
58. Perform a SPRI bead cleanup of the linearized plasmid as described in Step 26, using 1 volume of SPRI beads and eluting in 12 μL of nuclease-free water. Transfer the eluate to a new tube. Elution in nuclease-free water is important to achieve a high RNA yield from the in vitro transcription reaction.
59. Quantify the purified linearized plasmid by nanodrop and dilute it to 125 ng/μL.
   The linearized plasmid may be stored at −20° C. for extended periods of time before proceeding to in vitro transcription.
60. Prepare gRNA in vitro transcription reaction using the Promega T7 RiboMAX Express Large Scale RNA Production Kit (or equivalent) as follows. Multiple reactions from the same template plasmids can be performed to increase the gRNA yield. Incubate the reaction for 4-16 hours at 37° C.

| Reagent | Stock concentration | Volume (μL) |
|---|---|---|
| Linearized pT7-gRNA | 125 ng/μL | 8 |
| template from Step 59 | | |
| RiboMAX Express T7 Buffer | 2X | 10 |
| Enzyme Mix, T7 Express | 10X | 2 |
| Total | | 20 |

61. After incubation, add 1 μL of RQ1 DNase to the reaction to degrade the DNA template plasmid (RQ1 DNase is provided in the Promega in vitro transcription kit) and incubate at 37° C. for 15 minutes.
62. Perform a SPRI bead cleanup of the linearized plasmid as described in Step 26, using 3 volumes of SPRI beads and eluting in 50 μL of nuclease-free water. Preventing RNase contamination is important for achieving a high yield. Continue to clean the workspace and pipettes using RNase ZAP.
63. Refold the gRNA by heating it to 95° C. for 5 minutes, and then letting it cool to room temperature over 15 minutes.
64. Quantify the RNA by NanoDrop.
65. Distribute the gRNA into 10 μL aliquots and store at −80° C. gRNA aliquots can be stored at −80° C. for extended periods of time.

### Production of Nuclease-Containing Lysate

Cell culture and transfection should be performed for each nuclease (unless cotransfecting gRNAs, in which case, transfections must be carried out for each nuclease-gRNA pair). Transfections can be executed in parallel.
66. Cell culture and transfection; culturing, passaging, and seeding HEK 293 Ts. Culture the cells in HEK 293T culture medium (as described in the materials section) at 37° C. and 5% CO₂ in 150-mm culture dishes. Cells should be split every 48-72 hours, do not let them exceed 95% confluency. To passage the cells, discard the medium and rinse gently with 10 mL of PBS.

Add 3 mL of pre-warmed trypsin and incubate at 37° C. for approximately 5 minutes. Add 22 mL of pre-warmed media to quench trypsin and suspend the cells by pipetting. Count the cells and seed approximately 5×10⁶ or 2.5×10⁶ cells for 2 or 3 days of growth, respectively, in a total volume of 25 mL in a 150-mm culture dish. To seed HEK 293T cells in 24-well plates for next-day transfection, seed 1.5×10⁵ cells per well in 500 μL of HEK 293T culture medium per well. HEK 293 Ts can easily detach from the plate. Pipette PBS onto the side of the culture dish rather than directly onto the cells when passaging.
67. Approximately 24-hours after seeding cells in 24-well plates, prepare the nuclease expression plasmids for transfection using TransIT-X2 transfection reagent. The following conditions result in robust expression of a human codon optimized pCMV-T7-SpCas9-P2A-EGFP construct (RTW3027). If a U6 promoter gRNA expression plasmid is included in the transfection, it should be provided in excess relative to the nuclease expression plasmid such that gRNA is not limiting. In this case, a purified gRNA will not be required until Step 79 during the cleavage reactions.

| Reagent | Stock concentration | Volume (μL) |
|---|---|---|
| Nuclease expression plasmid | ~500 ng/μL | 1 |
| (e.g. RTW3027) | | |
| TranslT-X2 (Mirus) | | 1.5 |
| OptiMEM | | 22.5 |

The transfection mix should be added to cells within 30 minutes following the mixing of TransIT-X2 with OptiMEM and DNA for optimal transfection efficiency.
68. Gently mix the transfection solution and incubate at room temperature for 15 minutes.
69. Gently add the transfection solution dropwise onto the cells seeded in 24-well plates in Step 66 and mix by tilting the plate. Allow the cells to continue to grow for approximately 48-hours.
70. Nuclease-containing lysate preparation. Approximately 48-hours post-transfection, prepare fresh lysis buffer as described in the Materials section.
71. Prepare a fluorescein standard curve from a 2.5 mM Fluorescein dye stock solution as follows. Pipette carefully and mix well to ensure dilutions are accurate.

| Standard | Stock concentration | Preparation |
|---|---|---|
| Dilution A | 25 μM | 10 pL of 2.5 mM stock + |
| | | 990 pL of lysis buffer |
| 1 | 10 μM | 40 pL of Dilution A + |
| | | 60 pL of lysis buffer |
| 2 | 5 μM | 50 pL of Standard 1 + |
| | | 50 pL of lysis buffer |
| 3 | 2.5 μM | 50 pL of Standard 2 + |
| | | 50 pL of lysis buffer |
| 4 | 1.25 μM | 50 pL of Standard 3 + |
| | | 50 pL of lysis buffer |
| 5 | 625 nM | 50 pL of Standard 4 + |
| | | 50 pL of lysis buffer |
| 6 | 312.5 nM | 50 pL of Standard 5 + |
| | | 50 pL of lysis buffer |
| 7 | 156.25 nM | 50 pL of Standard 6 + |
| | | 50 pL of lysis buffer |
| 8 | 78.13 nM | 50 pL of Standard 7 + |
| | | 50 pL of lysis buffer |
| 9 | 39.06 nM | 50 pL of Standard 8 + |
| | | 50 pL of lysis buffer |
| 10 | 19.53 nM | 50 pL of Standard 9 + |
| | | 50 pL of lysis buffer |
| 11 | 9.77 nM | 50 pL of Standard 10 + |
| | | 50 pL of lysis buffer |
| 12 | 0 | 100 pL of lysis buffer |

72. Discard the media from the transfection plates from Step 69 and immediately add 100 μL of pre-chilled lysis buffer to each well. A smaller volume of lysis buffer can be used to concentrate lysates, if necessary. Pipette gently to mix the mixture of cells and lysis buffer, then cover the plates with an adhesive aluminum seal and gently rock at 4° C. for approximately 10 minutes. The lysate should be kept on ice or at 4° C. as soon as lysis buffer is added unless otherwise noted.
73. Transfer the lysates to a 96-well plate on ice. Note the plate layout as this sample layout will be maintained into the library preparation steps (see FIG. 19). Maintaining a consistent and logical sample layout will facilitate rapid library preparation and identification of samples.
74. Use a 12-channel multichannel to mix the samples and transfer 20 μL of each sample to wells of a 384-well black microplate. For standards 1-12, transfer 20 μl of each standard to the 384-well black microplate. Perform all measurements in duplicate and average the replicates. To use a smaller volume of lysate for the quantification (rather than 20 μL), mix 10 μL of lysate with 10 μL of lysis buffer in the 384-well plate. Account for any dilutions when determining the concentration of the lysate.
75. On a fluorescence plate reader, such as a DTX 880 Multimode Plate Reader (Beckman Coulter), set λₑₓ=485 nm and λₑₘ=535 nm and measure fluorescence of the 384-well plate (including samples and standards).
76. Generate a standard curve from the fluorescence readings of the standards. Determine the linear relationship between fluorescein concentration and fluorescence intensity by linear regression. Exclude any standards with fluorescence intensities that fall outside the linear range of the instrument.
77. Normalize all lysates by diluting samples from Step 73 to the desired concentration with lysis buffer and mix gently. The optimal concentration may require optimization for different Cas enzymes. For example, with SpCas9 nuclease, a lysate concentration corresponding to 150 nM fluorescein dye is recommended for in vitro cleavage reactions, which should lead to complete cleavage of substrates harboring targetable PAMs and a range of activities across non-canonical PAM substrates throughout the timecourse reaction. Alternatively, for SpCas9 base editors, a concentration corresponding to 600 nM fluorescein dye is recommended.
   We recommend optimizing the in vitro cleavage reaction conditions, particularly the lysate concentration and timepoint selection, based on the performance of well-studied CRISPR-Cas tools, such as SpCas9 for 3′ PAM substrate libraries and AsCas12a for 5′ PAM substrate libraries. A small-scale pilot HT-PAMDA experiment with a positive control can be performed to ensure that assay conditions are tuned to recapitulate the known performance of the CRISPR-Cas enzyme in the genome editing application of interest.
78. Aliquot normalized lysates into 96-well plates with approximately 10 μL of lysate per well in each plate. Store at −80° C. until use for in vitro cleavage reactions. The activity of the Cas protein contained in the lysate can be assayed by performing in vitro cleavage reactions on plasmid or linear DNA substrates harboring a target site corresponding to the gRNA(s) from Step 65. For in vitro cleavage reactions, follow the steps described below.
   Lysates can be stored at −80° C. for extended periods of time.

### Timecourse In Vitro Cleavage Reactions

This procedure should be carried out for each linearized library harboring randomized PAMs from Step 27 (henceforth referred to as “substrate libraries”). All steps should be performed with care to avoid cross-contamination.
79. Thaw the substrate library from Step 27, in vitro transcribed gRNA(s) from Step 65, and lysates from Step 78 on ice. Dilute the substrate library and gRNAs to the appropriate stock concentrations with nuclease-free water as follows.

| Reagent | Stock concentration |
|---|---|
| gRNA | 2.5 pM |
| Substrate library | 25 nM |

80. Dilute the 25 nM substrate library from Step 79 in water and cleavage buffer to generate the library working solution (4.5 nM substrate library) as follows. Dilute enough for all reactions and aliquot the solution into 8-strip tubes, with at least 9.625 μL per tube, to facilitate multichannel pipetting in Step 83. Prepare and aliquot sufficient excess solution to ensure the full 9.625 μL can be transferred in Step 83.

| | | | For 3.5x | |
|---|---|---|---|---|
| | | | reaction (3 | |
| | | Volume | timepoints | |
| | | per | with excess | |
| | Stock | timepoint | volume, | Final |
| Reagent | concentration | (μL) | in μL) | concentration |
| Substrate library | 25 nM | 0.5 | 1.75 | 4.5 nM |
| Cleavage buffer | 10X | 0.5 | 1.75 | 1.8X |
| Nuclease-free water | | 1.75 | 6.125 | |
| Total (per reaction) | | 2.75 | 9.625 | |

81. Prepare the reaction stop buffer as follows and aliquot 5 μL per well into 96-well plates, one plate per timepoint, at room temperature (FIG. 19). Label the plates.

| Reagent | Volume (μL) | Final concentration |
|---|---|---|
| Proteinase K (20 mg/ml) | 0.5 | 2 mg/ml |
| 0.5 M EDTA, pH 8.0 | 0.5 | 50 mM |
| Nuclease-free water | 4 | |
| Total (per reaction) | 5 | |

82. Mix the lysate from Step 27 (thawed in Step 79) and gRNA from Step 79 as follows in 8-strip tubes in a thermal cycler at 37° C., mix gently by pipetting, and let the Cas enzymes and gRNAs complex for between 3 to 15 minutes. Place the 8-strip tubes containing the 4.5 nM substrate library from Step 80 in the thermal cycler to warm the solution to 37° C. (FIG. 19).

| | | | For 3.5x | |
|---|---|---|---|---|
| | | | reaction (3 | |
| | | Volume | timepoints | |
| | | per | with excess | |
| | Stock | timepoint | volume, | Final |
| Reagent | concentration | (μL) | in μL) | concentration |
| gRNA (from | 2.5 μM | 1 | 3.5 | 1.1 μM |
| Step 79) | | | | |
| Lysate (from | 150 nM | 1.25 | 4.375 | 83 nM |
| Step 27) | (Fluorescein) | | | Fluorescein |
| Total | | | 7.875 | |

83. For each reaction ad 9.625 μL substrate library DNA (from Step to 7.875 μL of the lysate-gRNA mixture (from Step 82) with a multichannel pipette as follows and mix gently by pipetting (FIG. 19). Start up to 12 reactions at once using the multichannel pipette. Immediately start a timer.

| | | | For 3.5x | |
|---|---|---|---|---|
| | | | reaction (3 | |
| | | | timepoints | |
| | | Volume | with | |
| | | per | excess | |
| | Initial | timepoint | volume, in | Final |
| Reagent | concentration | (μL) | μL) | concentration |
| RNP | 1.1 μM gRNA, | 2.25 | 7.875 | 0.5 μM gRNA, |
| mixture | 83 nM | | | 37.5 nM |
| (step 82) | Fluorescein | | | Fluorescein |
| | (lysate) | | | (lysate) |
| Substrate | 4.5 nM substrate | 2.75 | 9.625 | 2.5 nM |
| library | library, 1.8X | | | substrate |
| working | cleavage buffer | | | library, 1X |
| solution | | | | cleavage |
| (step 80) | | | | buffer |
| Total | | 5 | 17.5 | |

84. At each timepoint, terminate reaction aliquots by transferring 5 μL from the reaction mixture in the thermal cycler (Step 83) into 5 μL of the pre-aliquoted reaction stop buffer in 96-well plates from Step 81 at room temperature as follows using the multichannel pipette. Mix the stop buffer and reaction mixture by pipetting.

| Reagent | Volume per timepoint (μL) |
|---|---|
| Reaction mixture (Step 83) | 5 |
| Reaction stop solution (Step 81) | 5 |

Stagger sets of 12 reactions to save time. For example, with timepoints of 1, 8, and 32 minutes, stagger four sets of 12 reactions for a total of 48 reactions simultaneously as follows:

| Reaction | | Start time | Timepoint 1 | Timepoint 2 | Timepoint 3 |
|---|---|---|---|---|---|
| set | Samples | (min) | (min) | (min) | (min) |
| 1 | 1-12 | 0 | 1 | 8 | 32 |
| 2 | 13-24 | 2 | 3 | 10 | 34 |
| 3 | 25-36 | 4 | 5 | 12 | 36 |
| 4 | 37-48 | 6 | 7 | 14 | 38 |

85. Following completion of the in vitro cleavage timecourses, wait until all terminated reactions have incubated at room temperature for at least 20 minutes to facilitate complete digestion of the Cas proteins by Proteinase K.
86. Seal plates well with an aluminum adhesive seal and heat to 98° C. for 10 minutes in a thermal cycler to inactivate Proteinase K.
   Plates of terminated and Proteinase K inactivated reactions can be stored at −20° C. for extended periods of time until proceeding to library preparation.
87. OPTIONAL. If performing HT-PAMDA using lysates expressing CBEs or ABEs instead of nucleases, the following additional enzymatic steps must be performed after Step 86.
   For CBEs, convert cytosine to uracil deamination events to DSBs by adding USER enzyme and buffer to each reaction from Step 86 as follows. Incubate reactions at 37° C. for 1 hour.

| Reagent | Stock concentration | Volume (μL) |
|---|---|---|
| USER enzyme | 1,000 units/mL | 1 |
| NEB buffer 4 | 10X | 0.5 |
| Water | | 3.5 |
| Total (per reaction) | | 5 |

For ABEs, convert adenosine to inosine deamination events to DSBs by adding Endonuclease V and buffer as follows to each reaction from Step 86. Incubate reactions at 37° C. for 1 hour.

| Reagent | Stock concentration | Volume (μL) |
|---|---|---|
| Endonuclease V | 10,000 units/mL | 1 |
| NEB buffer 4 | 10X | 0.5 |
| Water | | 3.5 |
| Total (per reaction) | | 5 |

To stop the USER or Endonuclease V treatments of the CBE and ABE reactions, respectively, add 5 μL of Proteinase K solution (prepared as follows) and incubate at 37° C. for 15 minutes.

| Reagent | Stock concentration | Volume (μL) |
|---|---|---|
| Proteinase K | 20 mg/mL | 1 |
| NEB buffer 4 | 10X | 0.5 |
| Water | | 3.5 |
| Total (per reaction) | | 5 |

Heat inactivate the Proteinase by incubating at 98° C. or 10 minutes.
Plates of terminated and Proteinase K inactivated reactions can be stored at −20° C. for extended periods of time until proceeding to library preparation.

Library Preparation

PCR #1—Sample Barcoding
PCR #1 will amplify uncleaved substrates from the HT-PAMDA cleavage reactions. Barcoded primers bind to sequences adjacent to the randomized PAM of the libraries, and append sample barcodes and Illumina read 1 and 2 sequencing primer binding sites (FIGS. 18 and 19). All steps should be performed with care to avoid cross-contamination.
88. Thaw reagents including terminated and Proteinase K inactivated in vitro cleavage reactions from Step 86 for nucleases or Step 87 for CBEs and ABEs, the arrayed barcode primer plate (see Reaction Setup section), and PCR reagents.
89. Prepare PCRs for every in vitro cleavage reaction, including no-template negative controls, as follows. Aliquot the PCR solution into wells of 96-well PCR plates corresponding to the same sample layout from the cleavage reactions.
   OPTIONAL: If the untreated substrate library was not sequenced in Steps 29-48, an untreated substrate library sample should be included now.

| Reagent | Stock concentration | Volume (μL) |
|---|---|---|
| Q5 buffer | 5X | 5 |
| dNTPs | 10 mM each | 0.5 |
| Betaine | 5 M | 2.5 |
| MgCl2 | 50 mM | 1.5 |
| Q5 polymerase | 2,000 units/mL | 0.25 |
| Nuclease-free water | | 11.25 |
| Total (per reaction) | | 21 |

90. To prepare each PCR, combine 1.5 μL of terminated and inactivated cleavage reaction (from Step 86 for nucleases or Step 87 for CBEs and ABEs) as template, with 2.5 μL of sample barcoding primer pairs (prepared in an arrayed plate format, as described in the reagent setup section) and 21 μL PCR solution (from Step 89). For ease of sample handing and identification, maintain an identical layout across all plates (e.g. row A of the PCR plate is combined with row A cleavage reaction template and row A primers).

| Reagent | Stock concentration | Volume (μL) |
|---|---|---|
| PCR solution (Step 89) | | 21 |
| Terminated and inactivated | 1.25 nM (Step 86) or | 1.5 |
| cleavage | 0.83 nM (Step 87) | |
| reaction (template; Step 86 or 87) | substrate library | |
| HT-PAMDA barcoded primer pairs | 5 μM each | 2.5 |
| Total (per reaction) | | 25 |

Each treated sample must receive a unique sample barcode primer pair. Any primer pair can be used for the no-template control.

If the untreated substrate library will be sequenced, a unique primer pair must be used to barcode the sample. If the full set of 96 primer pairs are used for experimental samples, a unique primer pair may be created for the untreated control by using one of the extra P5 sample barcoding primers not included in the arrayed primer plate (see Table 1).
91. Run all PCRs with the following program.

| Cycle number | Denature | Anneal | Extend |
|---|---|---|---|
| 1 | 98° C., 2 min | | |
| 2-31 | 98° C., 10 s | 67° C., 10 s | 72° C., 10 s |
| 32 | | | 72° C., 1 min |

92. Confirm the generation of PCR amplicons by running the reactions on a capillary electrophoresis machine (as described in Step 32) or an agarose gel. The sample should have a single band with a size of 206 bp.

Repeat any PCRs that exhibit low or no evidence of amplification.

Pooling of PCR samples corresponding to single timepoints
93. All PCR samples from a given timepoint can be pooled by combining 2 μL of each reaction (this tube should contain 2 μL of every uniquely barcoded sample from that timepoint) (FIGS. 18 and 19). If three timepoints were used during the in vitro cleavage reactions, there should be three total pools after this stage. Mix all timepoint pools well. If multiple libraries bearing distinct spacer sequences were used in the in vitro cleavage reactions, the amplicons of samples from corresponding timepoints from these separate libraries can be pooled together (as they are later deconvoluted informatically following sequencing, due to the presence of distinct spacer sequences). For example, if 96 reactions were performed using separately barcoded Cas lysates from a given timepoint across 2 substrate libraries (for a total of 192 samples), the 192 samples from a given timepoint can be combined into a single ‘timepoint pool’ (see FIGS. 18 and 19).
   Use a multichannel pipette to facilitate sample pooling.
   If an untreated substrate library control will be sequenced, add 10 μL of the uniquely barcoded amplicon generated from the untreated substrate library control to one of the timepoint pools. Note which timepoint pool contains this untreated library control as the location of this library sample must be provided during data analysis. For this protocol, we will assume that the untreated library control is added to the sample pool for timepoint 3. If multiple substrate libraries with distinct spacer sequences were used, pool both untreated substrate library amplicons together into the same timepoint pool. Relative to the 2 μL of each nuclease-treated sample that is combined in each pool, a larger 10 μL volume of untreated substrate library amplicon is pooled to ensure sufficient read depth for the untreated sample, which is used to normalize all other samples in the analysis.
94. Purify 50 μL of each timepoint pool with SPRI beads (as described in Step 26) using 1.5 volumes of SPRI beads. Elute in 25 μL of nuclease-free water. Withhold the remainder of the timepoint pool; store at −20° C. for extended periods of time.
95. Treat 10 μL of each purified timepoint pool with Exonuclease I as follows to degrade residual PCR #1 primers. Set up the reactions in 8-strip tubes. Incubate the reactions at 37° C. for 1 hour and then heat to 80° C. for 20 minutes to inactivate Exonuclease 1.

| Reagent | Stock concentration | Volume (μL) |
|---|---|---|
| Purified timepoint pool (step 94) | | 10 |
| Exonuclease 1 buffer | 10X | 4 |
| Exonuclease 1 | 20,000 units/mL | 3 |
| Nuclease-free water | | 23 |
| Total (per reaction) | | 40 |

Exonuclease I digestion is necessary to prevent sample barcoding primer carryover into the next round of PCR, which can reduce barcoding fidelity by introducing erroneously barcoded samples into the final library.
96. Purify heat-inactivated Exonuclease I reactions with SPRI beads (as described in Step 26) using 1 volume of SPRI beads. Elute in 25 μL of TE buffer.
97. Quantify the purified pools by NanoDrop. If the samples are too dilute for accurate nanodrop quantification, more sensitive methods such as Qubit, QuantiFluor, or alternatives can be used.
98. In new 8-strip tubes, create a dilution of each timepoint pool for a final concentration of approximately 0.125 ng/μL and a volume of at least 2 μL. Withhold the remaining concentrated pool; store at −20° C. for extended periods of time.
   This dilution is intended to limit the extent of post-Exonuclease I treatment residual PCR #1 sample barcoding primer carryover into the next round of PCR.
   The timepoint pools can be stored at −20° C. for extended periods of time before proceeding to the second PCR.

PCR #2—Timepoint Barcoding
99. Thaw the PCR reagents and the plate of timepoint barcoding primers for the second barcoding PCR (see FIGS. 18 and 19).
100. Prepare the PCR master solution as follows, generating enough solution for each sample and a no-template control. Aliquot the PCR master solution into 8-strip tubes.

| Reagent | Stock concentration | Volume (μL) |
|---|---|---|
| Q5 buffer | 5X | 4 |
| dNTPs | 10 mM each | 0.4 |
| Q5 polymerase | 2,000 units/mL | 0.2 |
| Nuclease-free water | | 11.4 |
| Total (per reaction) | | 16 |

101. To each 16 μL PCR rom Step 100, add 2 μL of diluted (0.125 ng/μL) timepoint pool (from Step 98) as template and 2 μL of 5 μM unique timepoint barcoding primer pairs (as described in Reagent Setup).

| Reagent | Stock concentration | Volume (μL) |
|---|---|---|
| PCR master solution (Step 100) | | 16 |
| Cleavage reaction (template) (Step 98) | 0.125 ng/μL | 2 |
| timepoint barcoding primer pair | 5 pM each | 2 |
| Total (per reaction) | | 20 |

Each timepoint pool must receive a unique timepoint barcode primer pair.
102. Run all PCRs with the following program.

| Cycle number | Denature | Anneal | Extend |
|---|---|---|---|
| 1 | 98° C., 2 min | | |
| 2-11 | 98° C., 10 s | 65° C., 30 s | 72° C., 30 s |
| 12 | | | 72° C., 5 min |

103. Confirm amplification by running the reactions on a capillary electrophoresis machine (as described in Step 32) or an agarose gel. All samples except the negative control should have a single band of roughly equal intensity with a size of 279 bp.
104. Purify the reactions with SPRI beads as described in Step 26 using 1.5 volumes of SPRI beads. Elute in 30 μL of TE buffer.

Purified timepoint pool PCRs can be stored at −20° C. for extended periods of time until proceeding to library quantification.

Library Quantification
105. Quantify the purified timepoint pool libraries (from Step 104) with the Universal KAPA Illumina Library qPCR Quantification Kit as described in Steps 38-46.
106. Based on the qPCR quantification, combine all timepoint pools (FIG. 19) such that all samples are equally represented to create a 4 nM library with a volume of at least 30 μL.

Accurate dilution of the library is important for ensuring appropriate cluster density during sequencing.

The final 4 nM HT-PAMDA library (FIG. 19) can be stored at −20° C. for extended periods of time until proceeding to sequencing.

### Sequencing

107. Thaw the 4 nM HT-PAMDA library (Step 106), PhiX v3 sequencing control, and sequencing kit reagents.
108. Dilute the PhiX sequencing control v3 to 4 nM by adding 2 μL of the 10 nM PhiX stock to 3 μL of 10 mM Tris-HCl (pH 8.5) with 0.1% Tween 20 solution and mix.
109. Denature 5 μL of the 4 nM PhiX solution by adding 5 μL of freshly prepared 0.2 N NaOH. Vortex briefly to mix, centrifuge at approximately 300×g for 1 minute, and incubate at room temperature for 5 minutes. After incubation, add 5 μL of 200 mM Tris-HCl (pH 8.0) and mix.
110. Denature 5 μL of the 4 nM HT-PAMDA library by adding 5 μL of freshly prepared
0.2 N NaOH. Vortex briefly to mix, centrifuge at approximately 300×g for 1 minute, and incubate at room temperature for 5 minutes. After incubation, add 5 μL of 200 mM Tris-HCl (pH 8.0) and mix.
111. Dilute the denatured PhiX from Step 109 and HT-PAMDA library from Step 110 by separately adding 985 μL of HT1 buffer (provided in the Illumina sequencing kit) to each and mixing. The resulting PhiX sample and HT-PAMDA library are both 20 μM.
112. Prepare the loading solution by combining the HT-PAMDA library and PhiX in appropriate ratios as follows, using the concentration and volume recommendations below:

| | 20 pM HT- | | | Total |
|---|---|---|---|---|
| | PAMDA library | 20 pM PhiX | HT1 buffer | volume |
| System (kit) | volume (μL) | volume (μL) | volume (μL) | (μL) |
| MiSeq | 300 | 75 | 225 | 600 |
| NextSeq 500/550 | 32.5 | 19.5 | 1248 | 1300 |
| (Mid Output Kit | | | | |
| v2.5) | | | | |
| NextSeq 500/550 | 39 | 23.4 | 1237.6 | 1300 |
| (High Output Kit | | | | |
| v2.5) | | | | |

Properly mix the resulting loading solution.

The HT-PAMDA library has low nucleotide diversity. Two-color sequencing systems like the NextSeq are especially sensitive to over-clustering with low nucleotide diversity libraries. For this reason, we recommend loading below Illumina's recommended library concentrations for the NextSeq system and using a high proportion of PhiX control (to improve nucleotide diversity). We recommend the following loading concentrations for the MiSeq and NextSeq:

| | | PhiX | Total |
|---|---|---|---|
| | HT-PAMDA library | concentration | concentration |
| System (kit) | concentration (pM) | (pM) | (pM) |
| MiSeq | 10 | 2.5 | 12.5 |
| NextSeq 500/550 | 0.5 | 0.3 | 0.8 |
| (Mid Output Kit | | | |
| v2.5) | | | |
| NextSeq 500/550 | 0.6 | 0.36 | 0.96 |
| (High Output Kit | | | |
| v2.5) | | | |

113. Add the complete volume of the loading solution (600 μL for the MiSeq or 1300 μL for the NextSeq) to the well indicated on the reagent cartridge for library loading.

Load the sequencer following standard protocols in the Illumina system manual and sequence the libraries with the following options: For NextSeq, put the instrument in “Manual Run Mode” (also called “Standalone Mode” prior to NextSeq Control Software 4.0). For the MiSeq, complete the run setup with the “Manual” option. Enter the number of cycles to meet the following minimum requirements, as follows:

| Read | Cycles |
|---|---|
| Read 1 | ≥65 |
| Read 2 | ≥10 |
| Index 1 | 8 |
| Index 2 | 8 |

### Analysis

114. Perform demultiplexing of the run to generate fastq files as described in Steps 50-52.
115. Navigate to the HT-PAMDA directory installed in Step 53 and repeat Step 54 to launch the HT-PAMDA virtual environment.
116. Enter the required inputs and run analysis pipeline. The analysis pipeline outputs CSV files and heatmap representations of PAM preference. Check the outputs for positive and negative control samples to verify the success of the experiment.

### Results.

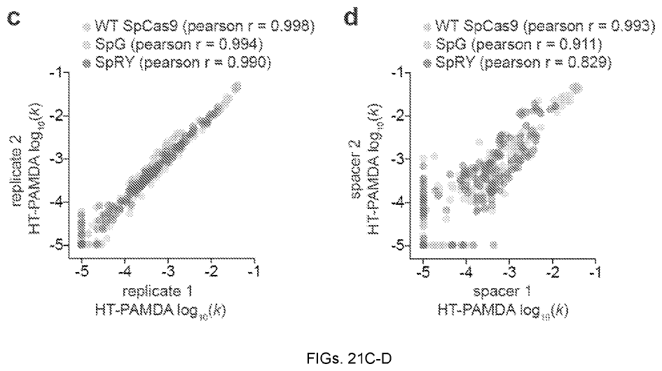

Deep sequencing of the randomized PAM libraries following library construction but prior to in vitro cleavage reactions ensures adequate representation of all PAMs. Additionally, the composition of the substrate library serves as the zero-timepoint sample for subsequent experiments. Library composition for two of our 3′ PAM substrate libraries is provided in the GitHub repository as a reference to compare user-constructed libraries. Ideally, all PAMs will have similar representation in the untreated substrate library; for analysis of an NNNN PAM window from the library, there are 256 possible PAM sequences that will have an average representation of 0.3906% of the library (FIG. 21a).

Control samples and replicates provide quality control metrics for an HT-PAMDA experiment. Well-characterized CRISPR nucleases for mammalian genome editing applications including SpCas9 and AsCas12a for 3′ and 5′ PAMs, respectively, can ensure appropriate assay performance to infer activities in mammalian cells. Raw read counts of each PAM from a given timepoint can verify the success of an HT-PAMDA experiment; the PAM read count distribution of the no-guide control should not deviate from that of the untreated substrate library, while experimental samples should show depletion and enrichment of sequences that are consistent with the expected PAM profile (FIG. 21a). Normalized read counts at each timepoint should reveal the expected depletion patterns of known canonical and non-canonical PAMs. For example, WT SpCas9 should deplete canonical NGG PAMs at early timepoints, weaker non-canonical PAMs such as NAG and NGA at later timepoints, and should not alter the normalized fraction of non-targetable PAMs like NCC (FIG. 21b). In the heatmap representation, rate constants of PAM depletion (HT-PAMDA log₁₀ (k)) are depicted by color scale indicating no depletion to fast depletion (from white to dark blue, respectively; FIG. 21b). Importantly, the heatmap scale reflects absolute activity, enabling comparison of activity between nucleases represented by different heatmaps (FIG. 20d). Technical replicates of the same PAM library should be highly reproducible (FIG. 21c), and replicates of randomized PAM libraries with distinct spacer sequences should be consistent unless the PAM preference of a nuclease is strongly influenced by spacer sequence (FIG. 21d).

### REFERENCES

1. Komor, A. C., Kim, Y. B., Packer, M. S., Zuris, J. A. & Liu, D. R. Programmable editing of a target base in genomic DNA without double-stranded DNA cleavage. Nature 533, 420-4 (2016).
2. Gaudelli, N. M. et al. Programmable base editing of A⋅T to G⋅C in genomic DNA without DNA cleavage. Nature 551, 464-471 (2017).
3. Sternberg, S. H., Redding, S., Jinek, M., Greene, E. C. & Doudna, J. A. DNA interrogation by the CRISPR RNA-guided endonuclease Cas9. Nature 507, 62-7 (2014).
4. Jiang, W., Bikard, D., Cox, D., Zhang, F. & Marraffini, L. A. RNA-guided editing of bacterial genomes using CRISPR-Cas systems. Nat Biotechnol 31, 233-9 (2013).
5. Jinek, M. et al. A Programmable Dual-RNA-Guided DNA Endonuclease in Adaptive Bacterial Immunity. Science 337, 816-821 (2012).
6. Kleinstiver, B. P. et al. Engineered CRISPR-Cas12a variants with increased activities and improved targeting ranges for gene, epigenetic and base editing. Nat Biotechnol 37, 276-282 (2019).
7. Gao, L. et al. Engineered Cpf1 variants with altered PAM specificities. Nat Biotechnol 35, 789-792 (2017).
8. Kleinstiver, B. P. et al. Broadening the targeting range of Staphylococcus aureus CRISPRCas9 by modifying PAM recognition. Nat Biotechnol 33, 1293-1298 (2015).
9. Kleinstiver, B. P. et al. Engineered CRISPR-Cas9 nucleases with altered PAM specificities.

Nature 523, 481-5 (2015).
10. Kleinstiver, B. P. et al. High-fidelity CRISPR-Cas9 nucleases with no detectable genome-wide off-target effects. Nature 529, 490-5 (2016).
11. Rees, H. A. & Liu, D. R. Base editing: precision chemistry on the genome and transcriptome of living cells. Nat Rev Genet 19, 770-788 (2018).
12. Tsai, S. Q. et al. GUIDE-seq enables genome-wide profiling of off-target cleavage by CRISPR-Cas nucleases. Nat Biotechnol 33, 187-97 (2014).
13. Kleinstiver, B. P. et al. Genome-wide specificities of CRISPR-Cas Cpf1 nucleases in human cells. Nat Biotechnol 34, 869-74 (2016).
14. Tsai, S. Q. & Joung, K. J. Defining and improving the genome-wide specificities of CRISPRCas9 nucleases. Nat Rev Genet 17, 300-312 (2016).
15. Tsai, S. Q. et al. CIRCLE-seq: a highly sensitive in vitro screen for genome-wide CRISPRCas9 nuclease off-targets. Nat Methods 14, 607-614 (2017).
16. Chen, J. S. et al. Enhanced proofreading governs CRISPR-Cas9 targeting accuracy. Nature
   550, 407-410 (2017).
17. aymaker, I. et al. Rationally engineered Cas9 nucleases with improved specificity. Sci New York N Y 351, 84-8 (2015).
18. Hsu, P. D. et al. DNA targeting specificity of RNA-guided Cas9 nucleases. Nat Biotechnol
   31, 827-32 (2013).
19. Zetsche, B. et al. Cpf1 is a single RNA-guided endonuclease of a class 2 CRISPR-Cas system. Cell 163, 759-71 (2015).
20. Kim, D. et al. Genome-wide analysis reveals specificities of Cpf1 endonucleases in human cells. Nat Biotechnol 34, 863-8 (2016).
21. Rohland, N. & Reich, D. Cost-effective, high-throughput DNA sequencing libraries for multiplexed target capture. Genome Res 22, 939-46 (2012).
22. Anders, C., Niewoehner, O., Duerst, A. & Jinek, M. Structural basis of PAM-dependent target DNA recognition by the Cas9 endonuclease. Nature 513, 569-73 (2014).
23. Hu, J. H. et al. Evolved Cas9 variants with broad PAM compatibility and high DNA specificity. Nature 556, 57-63 (2018).
24. Nishimasu, H. et al. Engineered CRISPR-Cas9 nuclease with expanded targeting space.

Science 361, 1259-1262 (2018).
25. Hirano, S., Nishimasu, H., Ishitani, R. & Nureki, O. Structural Basis for the Altered PAM Specificities of Engineered CRISPR-Cas9. Mol Cell 61, 886-94 (2016).
26. Jinek, M. et al. A Programmable Dual-RNA-Guided DNA Endonuclease in Adaptive Bacterial Immunity. Science 337, 816-821 (2012).
27. Jiang, W., Bikard, D., Cox, D., Zhang, F. & Marraffini, L. A. RNA-guided editing of bacterial genomes using CRISPR-Cas systems. Nat Biotechnol 31, 233-9 (2013).
28. Kleinstiver, B. P. et al. Engineered CRISPR-Cas9 nucleases with altered PAM specificities.

Nature 523, 481-5 (2015).
29. Suzuki, K. et al. In vivo genome editing via CRISPR/Cas9 mediated homology-independent targeted integration. Nature 540, 144-149 (2016).
30. Wu, Y. et al. Highly efficient therapeutic gene editing of human hematopoietic stem cells.

Nat Med 25, 776-783 (2019).
31. Kleinstiver, B. P. et al. High-fidelity CRISPR-Cas9 nucleases with no detectable genome-wide off-target effects. Nature 529, 490-5 (2016).

1. Providing a plurality of individual discrete samples comprising populations of cells, preferably mammalian cells, preferably human cells, wherein each population of cells overexpresses both (i) a single genome engineering protein or a variant thereof and (ii) a reporter protein, wherein (i) and (ii) are expressed in a known ratio, preferably 1:1, in the samples;
lysing the cells to release the proteins;
normalizing levels of the genome engineering proteins or variants thereof based on levels of the reporter protein;
allowing the genome engineering proteins or variants thereof to combine with a guide RNA under conditions sufficient to form ribonucleoprotein complexes in each sample;
contacting each sample with a plurality of analysis substrates, under conditions sufficient for the genome engineering protein or variant thereof to act on one or more of the substrates;
determining levels of each of the analysis substrate in each sample at a plurality of times; and
calculating rate of depletion or enrichment of each of the analysis substrates from each sample.

2. The method of claim 1, wherein the genome engineering protein is a nuclease, base editor, or other protein that can alter DNA.

3. The method of claim 2, wherein the genome engineering protein can alter the genome of a living cell or genomic DNA in vitro)

4. The method of claim 1, wherein (i) and (ii) are expressed in a known ratio, e.g., 1:1 ratio, from a single nucleic acid construct, preferably a construct comprising a viral 2A sequence in between sequences encoding (i) and (ii), or a direct fusion between sequences encoding (i) and (ii) by a peptide linker.

5. The method of claim 1, wherein the reporter proteins are fluorescent.

6. The method of claim 5, wherein expression levels of the reporter proteins is determined by spectrophotometry, image analysis, or other methods to quantify the levels of fluorescence from the reporter protein.

7. The method of claim 1, wherein each different genome engineering protein or variant thereof is expressed in an identified discrete individual population of cells in a single well of a multi-well plate.

8. The method of claim 7, wherein a normalized amount of each genome engineering protein is transferred to a second multiwell plate.

9. The method of claim 1, wherein the genome engineering protein is or comprises a CRISPR nuclease, is mixed with a guide RNA to form ribonucleoprotein complexes, and is contacted with a population of analysis substrates, each comprising a spacer sequence and a PAM sequence, wherein the population comprises analysis substrates having a plurality of spacer sequences, or plurality of PAM sequences, or both.

10. The method of claim 1, wherein the genome engineering protein is or comprises a cytosine base editor, is mixed with a guide RNA to form ribonucleoprotein complexes, is contacted with a population of analysis substrates, each comprising a spacer sequence and a PAM sequence, wherein the population comprises analysis substrates having a plurality of spacer sequences, or plurality of PAM sequences, or both, and is contacted with an enzyme that converts C-to-U deamination events to double-strand breaks when they co-occur with SpCas9-HNH domain mediated DNA nicks.

11. The method of claim 1, wherein the genome engineering protein is or comprises a adenine base editor, is mixed with a guide RNA to form ribonucleoprotein complexes, is contacted with a population of analysis substrates, each comprising a spacer sequence and a PAM sequence, wherein the population comprises analysis substrates having a plurality of spacer sequences, or plurality of PAM sequences, or both, and is contacted with an enzyme that converts a combination of a target strand nick and a non-target strand deamination event to a double strand break, e.g., Endonuclease V.

12. The method of claim 1, wherein the guide RNA is expressed in the cells or is added to the samples.

13. The method of claim 1, wherein the analysis substrates include identifying sequences, preferably 8-10 nt barcodes.

14. The method of claim 1, wherein determining levels of each of the analysis substrate in each sample at a plurality of times comprises using sequencing, detectably labeled probes, arrays, or hybridization methods.

15. The method of claim 1, wherein determining the rate of depletion of each analysis substrate from the population of analysis substrates over time is determined by modeling the depletion as exponential decay and determining the rate constant of depletion for each analysis substrate.

16. The method of claim 15, further comprising identifying analysis substrates that are depleted at a faster rate as substrates for the genome engineering protein.


SEQUENCE LISTING (14 sequences)

SEQ ID NO: 1 - DNA, 15 bp, Artificial Sequence
  Description of Artificial Sequence Synthetic primer
cctcgtgacctgcgc

SEQ ID NO: 2 - DNA, 67 bp, Artificial Sequence
  Description of Artificial Sequence Synthetic oligonucleotide
  modified_base (36)..(43): a, c, t, g, unknown or other
  modified_base (46)..(48): a, c, t, g, unknown or other
gcaggaattcgggaggggcacgggcagcttgccggnnnnnnnnctnnngcgcaggtcacgaggcatg

SEQ ID NO: 3 - DNA, 67 bp, Artificial Sequence
  Description of Artificial Sequence Synthetic oligonucleotide
  modified_base (36)..(43): a, c, t, g, unknown or other
  modified_base (46)..(48): a, c, t, g, unknown or other
gcaggaattcggagggtcgccctcgaacttcacctnnnnnnnnctnnngcgcaggtcacgaggcatg

SEQ ID NO: 4 - DNA, 69 bp, Artificial Sequence
  Description of Artificial Sequence Synthetic oligonucleotide
  modified_base (13)..(15): a, c, t, g, unknown or other
  modified_base (18)..(27): a, c, t, g, unknown or other
agaccggaattcnnngtnnnnnnnnnnggaatcccttctgcagcacctgggcgcaggtcacgaggcatg

SEQ ID NO: 5 - DNA, 69 bp, Artificial Sequence
  Description of Artificial Sequence Synthetic oligonucleotide
  modified_base (13)..(15): a, c, t, g, unknown or other
  modified_base (18)..(27): a, c, t, g, unknown or other
agaccggaattcnnngtnnnnnnnnnnctgatggtccatgtctgttactcgcgcaggtcacgaggcatg

SEQ ID NO: 6 - DNA, 20 bp, Artificial Sequence
  Description of Artificial Sequence Synthetic oligonucleotide
gggcacgggcagcttgccgg

SEQ ID NO: 7 - DNA, 20 bp, Artificial Sequence
  Description of Artificial Sequence Synthetic oligonucleotide
gtcgccctcgaacttcacct

SEQ ID NO: 8 - DNA, 20 bp, Artificial Sequence
  Description of Artificial Sequence Synthetic oligonucleotide
gggcacgggcagcttgccga

SEQ ID NO: 9 - DNA, 20 bp, Artificial Sequence
  Description of Artificial Sequence Synthetic oligonucleotide
gggcacgggcagcttgccgt

SEQ ID NO: 10 - DNA, 20 bp, Artificial Sequence
  Description of Artificial Sequence Synthetic oligonucleotide
gggcacgggcagcttgccag

SEQ ID NO: 11 - DNA, 20 bp, Artificial Sequence
  Description of Artificial Sequence Synthetic oligonucleotide
tggcacgggcagcttgccgg

SEQ ID NO: 12 - DNA, 49 bp, Artificial Sequence
  Description of Artificial Sequence Synthetic oligonucleotide
  modified_base (40)..(49): a, c, t, g or absent
gcgactttccctcactcctgctcggtgaatccgaaaacgnnnnnnnnnn

SEQ ID NO: 13 - DNA, 49 bp, Artificial Sequence; identical to SEQ ID NO: 12
  Description of Artificial Sequence Synthetic oligonucleotide
  modified_base (40)..(49): a, c, t, g or absent

SEQ ID NO: 14 - DNA, 49 bp, Artificial Sequence; identical to SEQ ID NO: 12
  Description of Artificial Sequence Synthetic oligonucleotide
  modified_base (40)..(49): a, c, t, g or absent